(19) United States
(12) Patent Application Publication
(10) Pub. No.: US 2010/0279990 A1
(43) Pub. Date: Nov. 4, 2010

(54) ANTIPROLIFERATIVE COMPOUNDS, COMPOSITIONS AND METHODS OF USE

(75) Inventors: Irit Gil-Ad, Herzeila (IL); Moshe Portnoy, Givat Shmuel (IL); Avraham Weizman, Tel-Aviv (IL); Liat Lomnitski, Givat Shmuel (IL)

Correspondence Address: LERNER, DAVID, LITTENBERG,;KRUMHOLZ & MENTLIK, 600 SOUTH AVENUE WEST, WESTFIELD, NJ, 07090, US

(73) Assignees: RAMOT AT TEL-AVIV UNIVERSITY LTD., Tel Aviv (IL); PERRIGO ISRAEL PHARMACEUTICALS LTD., Bnei Brak (IL)

(21) Appl. No.: 12/308,299

(22) PCT Filed: Jun. 8, 2007
(86) PCT No.: PCT/US2007/070747

§ 371 Date: Jul. 23, 2010

Related U.S. Application Data
(60) Provisional application No. 60/813,079, filed on Jun. 13, 2006.

(51) Int. Cl. A61K 31/56; C07C 215/42; A61K 31/137; A61P 35/00; A61P 17/00; A61P 17/06; A61P 35/02; A61P 19/02; A61P 29/00; C07D 405/12; A61K 31/4525

(52) U.S. Cl. 514/171; 564/308; 514/647; 546/197; 514/321

(57) ABSTRACT

The present invention provides a compound of the formula A-L-B (I) or a pharmaceutically acceptable salt, ester or prodrug thereof, wherein A is a psychotropic derivative; L is a linking group comprising two carbon atoms; and B is an alkyl, alkenyl, alkynyl or aralkyl comprising at least one substituent of the formula Q, wherein: the alkyl, alkenyl, alkynyl or aralkyl is optionally substituted with one or more halogens, hydroxyl, cyano, nitro, amino or thiol; and Q is OR⁶, OC(O)R⁶, C(O)R⁶ C(S)R⁶, CO²R⁶, C(O)SR⁶, C(O)NR₆R⁷, C(S)NR⁶R⁷, NR⁶R⁷, NR⁶C(O)R⁷, NR⁶C(S)R⁷, NR⁶C(O)NR⁷R⁸, NR⁶C(S)NR⁷R⁸, NR⁶SO²R⁷, NR⁶SO²NR⁷R⁸, SR⁶, SC(O)R⁶, SC(O)NR⁶R⁷, S(O)R⁶, SO²R⁶, SO²NR⁶R⁷, or NR⁶SO²R⁷R⁸. Also provided is a pharmaceutical composition that includes one or more compounds of the present invention, and methods of therapeutically using and processes for producing such compounds and compositions.

### BACKGROUND OF THE INVENTION

Psychotropics include drugs or agents that are typically employed for the therapy of psychiatric disorders such as schizophrenia and mood disorders. In general, psychotropic drugs interact with central and peripheral neurotransmitters and their receptors, such as serotoninergic, dopaminergic, α-adrenergic, cholinergic etc. Selective serotonin reuptake inhibitors (SSRIs), such as paroxetine, sertraline, and fluoxetine, are among the most commonly prescribed antidepressants and are considered highly effective and relatively safe.

The antiproliferative activity of some psychotropic drugs also has been described. For example, Silver et al. showed the inhibitory effect of anti-psychotic drugs, such as haloperidol, flupentixol, dopamine and fluphenazine on human neuroblastoma cell lines. See Silver et al., Society of Biological Psychiatry, 35, 824-826, (1999). Other studies have shown that phenothiazines have anti-proliferative effects on some tumor cells such as leukemic cells, melanoma, glioma and leukemia. See Nordenberg et al., Biochemical Pharmacology, 58, 1229-1239, (1999); Gil-Ad et al., J. Molecular Neuroscience, 22, 189-198, (2004). Another study has established that some antidepressants produce a differential apoptotic effect in rat glioma cells and in human neuroblastoma. See Levkovitz, Gil Ad, et al., J. Molecular Neuroscience, 27, 29 (2005). An important marker for apoptosis is the activation of caspase-3, a key mediator implicated in apoptosis in mammalian cells that belongs to the asparate-specific cysteinyl proteases or caspases. We have previously reported an increase in caspase-3 activity following exposure to some SSRIs in rat glioma and human neuroblastoma cells, and suggested the potential use of SSRIs (e.g., paroxetine and fluoxetine) and tricyclic antidepressants (e.g., clomipramine) for therapy of brain tumors. See Levkovitz, Gil Ad, et al., J. Molecular Neuroscience, 27, 29 (2005).

Some SSRIs, such as fluoxetine and paroxetine, have been demonstrated to have an antiproliferative effect on several human and mouse cancer cells, as well as on non-malignant cells and psoriasis. See US 2005/0013853. WO 2004/030618 describes a method for treating inflammation with a combination of SSRIs and corticosteroids, providing increased inhibition of proinflammatory cytokines. WO 01/66101 describes the systemic administration (e.g., oral and transdermal administration) of some monocyclic antidepressant drugs, commonly linked by their norepinephrine reuptake inhibiting activity, for the treatment of psoriasis. In addition, U.S. Pat. No. 5,104,858 describes sensitizing multidrug resistant (MDR) cells to anti-tumor agents by contacting the cells with certain phenothiazines and thioxanthenes.

One of the major obstacles in inducing responsiveness in cancer cells is the existence of efflux transporter P-glycoprotein (Pgp), which is linked to Multidrug resistant (MDR) genes. Pgp, which belongs to the ATP-binding cassette (ABC) family of transporter molecules and requires hydrolysis of ATP to run the transport mechanism, confers upon cancer cells the ability to resist lethal doses of certain cytotoxic drugs by pumping the drugs out of the cells and thus reducing their cytotoxicity. Pgp substrates may be endogenous (steroid hormones, cytokines) or exogenous (cytostatic drugs). In principle, Pgp-mediated drug resistance can be circumvented by treatment regimens that either exclude Pgp substrate drugs or include Pgp inhibitory agents.

In a recent report, Peer et al. provided evidence that the antidepressant (SSRI) fluoxetine synergistically enhanced responses to chemotherapy in human xenograft mouse tumor models, by inhibition of the MDR extrusion pump, Pgp. See Peer et al., Cancer Res., 64(20), 7562 (2004). A variety of antidepressants, including citalopram, fluoxetine, fluvoxamine, paroxetine, reboxetine, sertraline, and venlafaxine and their major metabolites, such as desmethylcitalopram, norfluoxetine, paroxetine-metabolite (paroxetine-M), and desmethylsertraline, have been identified as Pgp inhibitors at similar concentrations as quinidine, a classical Pgp inhibitor. These observations have been made in two models: cancer MDR1 cells'and a model for blood brain barrier. Sertraline, desmethylsertraline, and paroxetine were found to be among the most potent. See Weiss J., et al., J. Pharmacol. Exp. Ther. Apr., 305(1):197-204 (2003). US 2005/0013853 describes the use of topically applied or systemically administered psychotropics, other than fluoxetine, for sensitizing multidrug resistance cancerous skin cells, such as melanoma cells.

Some antidepressants have been reported to exhibit anticancer activity. For instance, clomipramine, imipramine, and citalopram were reported to induce apoptosis in myeloid leukemia HL-60 cells. See Xia et al., J. Biochem. Mol. Toxicol., 13, 338-347 (1999). In addition, the monocyclic serotonin reuptake inhibitors, fluoxetine and zimelidine, were reported to inhibit proliferation of prostate carcinoma cells. See Abdul et al., J. Urol., 154, 247-250 (1995). However, some studies observed an increase the development of fibrosarcoma, melanoma, and breast tumors from in vivo administration of fluoxetine and amitryptiline. See Brandes et al., Cancer Res., 52, 3796-3800 (1992). In addition, certain known tricyclic antidepressants and paroxetine have been reported to be associated with either an elevated risk of breast cancer or to be ineffective in some cases. See Cotterchio et al., Am. J. Epidemiol., 151, 951-7 (2000); Wang et al., J. Clin. Epidemiol., 54, 728-34 (2001).

The Mitogen Activation Protein Kinase (MAPK) pathway is of major importance in the regulation of proliferation and apoptosis mechanisms. One of the early signaling cascades, which has been shown to mediate apoptotic cell death in response to a variety of stressful stimuli, is the c-Jun-N-terminal kinase (JNK) pathway. See Dérijard et al., Cell, 76(6), 1025-37 (1994). Extensive evidence indicates that the INK pathway is activated to provide apoptosis in a variety of different cell-lines, such as PC12 cells. See Xia et al., Science, 270(5240), 1326-31 (1995). Moreover, p-c-Jun is a major end product of the JNK pathway activation.

It has also been shown that in a rat glioma (C6) cell-line, human neuroblastoma active antidepressants, such as paroxetine, fluoxetine, and clomipramine, induce a rapid increase in p-c-Jun, as an early signaling step in apoptosis. See Levkovitz, Gil-Ad et al., J. of Molecular Neuroscience, 27, 29 (2005). Thus it seems that early activation of the MAPK c-Jun-N-terminal kinase (JNK) pathway can be a marker for potential psychotropic derivatives with antiproliferative activity.

A need exists for psychotropic derivatives having improved antiproliferative activity, compositions containing such compounds, and methods of preparing and using such compounds and compositions. The present invention provides such compounds, compositions and methods.

### BRIEF SUMMARY OF THE INVENTION

The present invention provides a compound of the formula A-L-B (I), wherein A is represented by the formulae (A1), (A2), and (A3):

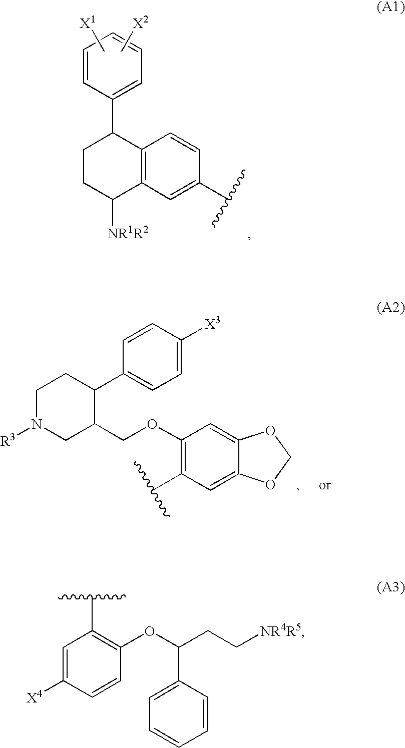

wherein: R¹, R², R³, R⁴ and R⁵ are the same or different and each is independently a hydrogen or alkyl; X¹ and X² are the same or different and each is independently a hydrogen, a halogen, haloalkyl, alkoxy, or a cyano; X³ is a hydrogen, alkyl, alkoxy, haloalkyl, a hydroxyl, a halogen, alkylthio, or an arylalkoxy; X⁴ is a halogen, haloalkyl, alkyl, alkoxy, or alkenyl; L is a linking group comprising two carbon atoms; and B is an alkyl, alkenyl, alkynyl or aralkyl comprising at least one substituent of the formula Q, wherein the alkyl, alkenyl, alkynyl or aralkyl is optionally substituted with one or more halogens, hydroxyl; cyano, nitro, amino, or thiol, and Q is OR⁶, OC(O)R⁶, C(O)R⁶, C(S)R⁶, CO₂R⁶, C(O)SR⁶, C(O)NR⁶R⁷, C(S)NR⁶R⁷, NR⁶R⁷, NR⁶C(O)R⁷, NR⁶C(S)R⁷, NR⁶C(O)NR⁷R⁸, NR⁶C(S)NR⁷R⁸, NR⁶SO₂R⁷, NR⁶SO₂NR⁷R⁸, SR⁶, SC(O)R⁶, SC(O)NR⁶R⁷, S(O)R⁶, SO₂R⁶, SO₂NR⁶R⁷, or NR⁶SO₂NR⁷R⁸, wherein R⁶, R⁷, and R⁸ are the same or different and each is independently a hydrogen, a C₁₋₆ alkyl, an aryl, an aralkyl, or a pharmaceutically acceptable solubility modifying group; or a salt, ester, or prodrug of the compound, which may include, e.g. a pharmaceutically acceptable salt, ester, etc. It will be appreciated that the compound of the formula A-L-B (I) includes geometric and optical isomers, e.g., diasteomers and mixtures thereof, enantiomers (e.g., a substantially pure enantiomer or an enantiomeric mixture), and molecules of the same general formula having any other suitable combination of chiral centers. The compound of the formula A-L-B (I) also includes, e.g., solvates, hydrates and polymorphs thereof.

Also provided, is a pharmaceutical composition comprising a pharmaceutically acceptable carrier and a therapeutically effective amount of at least one compound of the present invention.

The present invention further provides a method of treating a disease or disorder associated with abnormal or undesirable cell proliferation in a patient, wherein the method comprises administering to the patient a therapeutically effective amount of at least one compound of the present invention.

In addition, the present invention provides a method of treating cancer, wherein the method comprises administering to the patient a therapeutically effective amount of at least one compound of the present invention.

Also provided are processes of preparing compounds of the present invention. An exemplary process includes reacting a compound of the formula:

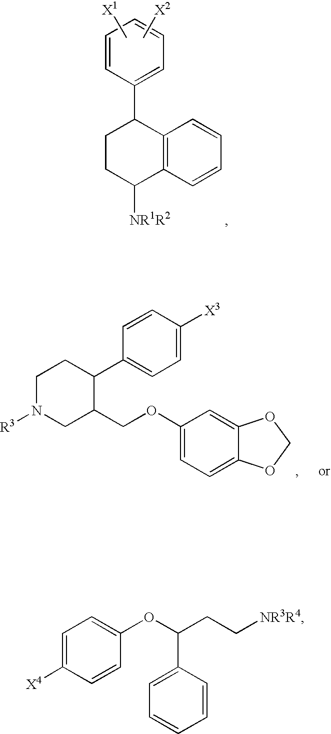

with a halogenating reagent to produce a halogenated compound of the formula:

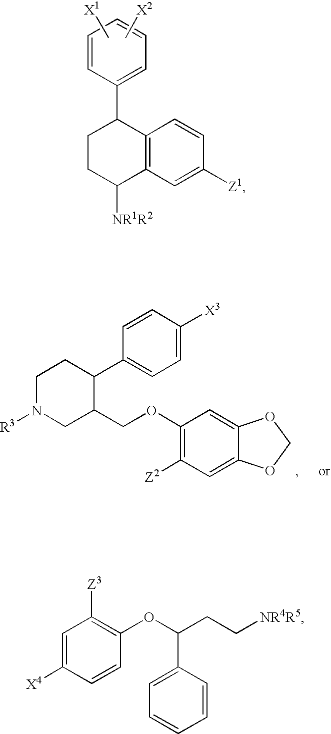

wherein, Z¹, Z², and Z³ are the same or different and are each a halogen, and coupling the halogenated compound with a compound of the formula L-B, wherein L is a linking group comprising a carbon-carbon triple bond and B is an alkyl, alkenyl, alkynyl or aralkyl such as, e.g., —(CH₂)ₙ(CHR⁹)ₘQ, —Ar(CH₂)ₙ(CHR⁹)ₘQ, —(CH₂)ₙAr(CHR⁹)ₘQ, or —(CH₂)ₙ(CHR⁹)ₘArQ wherein R¹-R⁹, X¹-X⁴, Ar, Q, m and n are as defined herein, to produce a coupling product comprising a carbon-carbon triple bond, optionally converting the carbon-carbon triple bond of the coupling product into a carbon-carbon double bond or carbon-carbon single bond, optionally introducing a pharmaceutically acceptable solubility modifying group to the coupling product, and optionally converting the coupling product into a pharmaceutically acceptable salt, ester, or prodrug, to produce a compound of the formula A-L-B (I) as defined herein.

Another exemplary process of the present invention includes regioselectively formylating a compound of the formula:

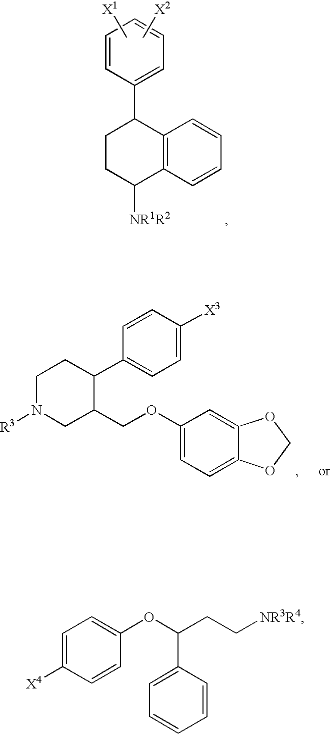

by reacting the compound with a formylating reagent (e.g., under suitable Vilsmeier-Haack conditions), to produce a formylated compound of the formula:

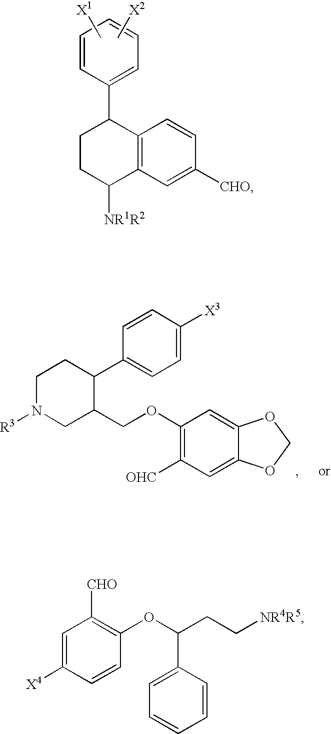

and reacting the formylated compound with a reagent capable of reacting with the formyl substituent (e.g., using a suitable a Wittig reagent or other aldehyde alkenylation reagent), to produce an alkenyl product of the formula:

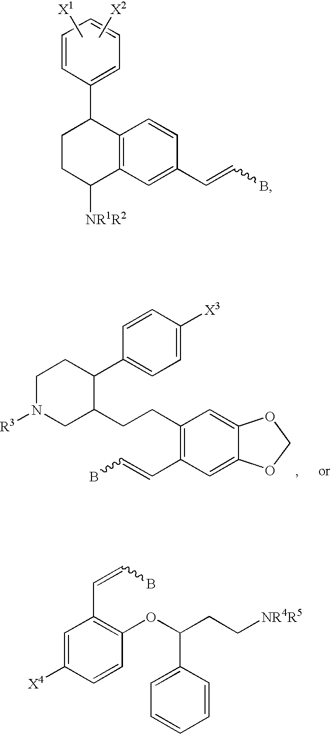

wherein B is as defined herein, and optionally converting the carbon-carbon double bond of the alkenyl product into a carbon-carbon single bond, optionally introducing a pharmaceutically acceptable solubility modifying group to the alkenyl product, and optionally converting the alkenyl product into a pharmaceutically acceptable salt, ester, or prodrug, to produce a compound of the formula A-L-B (I) as defined herein.

### BRIEF DESCRIPTION OF THE DRAWINGS

FIG. 1A illustrates the effect of an exemplary compound of the present invention on the viability of human Jurkat lymphoma cells.

FIG. 1B illustrates the effect of exemplary compounds of the present invention on the viability of human Jurkat lymphoma cells.

FIG. 1C illustrates the effect of an exemplary compound of the present invention on the viability of human Jurkat lymphoma cells.

FIG. 2A illustrates the effect of an exemplary compound of the present invention on the viability of human colon carcinoma cells (HT29).

FIG. 2B illustrates the effect of an exemplary compound of the present invention on the viability of human colon carcinoma cells (HT29).

FIG. 3A illustrates the effect of an exemplary compound of the present invention on the viability of Con-A activated naive mouse splenocytes.

FIG. 3B illustrates the effect of an exemplary compound of the present invention on IFN γ secretion in Con-A activated naive mouse splenocytes.

FIG. 4 illustrates the effect of an exemplary compound of the present invention on the viability of PHA activated healthy human T cells.

FIG. 5 illustrates the effect of an exemplary compound of the present invention on the viability of HaCat cells.

FIG. 6 illustrates western blot data corresponding to c-jun and p-c-jun expression in HaCat cells treated with an exemplary compound of the present invention.

FIG. 7A illustrates the effect of an exemplary compound of the present invention on the viability of mouse resistant colon carcinoma LS1034 cells.

FIG. 7B illustrates the effect of an exemplary compound of the present invention on the viability of LS1034 human colon MDR carcinoma cells.

FIG. 8A illustrates the effect of an exemplary compound of the present invention on Caspase 3 activity in human Jurkat lymphoma cells.

FIG. 8B illustrates the effect of an exemplary compound of the present invention on Caspase 3 activity of LS1034 human colon MDR carcinoma cells.

FIG. 9 illustrates the effect of an exemplary compound of the present invention on the viability of human glioma U87 cells.

FIG. 10 illustrates western blot data corresponding to BCL2 expression in human Jurkat lymphoma cells treated with an exemplary compound of the present invention.

FIG. 11 illustrates the acute toxicity of an exemplary compound of the present invention on male BalbC mice.

FIG. 12 illustrates the effect of the combination of an exemplary compound of the present invention and the chemotherapeutic agent doxorubicin on the viability of Jurkat lymphoma cells.

FIG. 13A illustrates the effect of the corticosteroid dexamethasone, the antidepressants paroxetine and sertraline, and an exemplary compound of the present invention on viability of human Jurkat lymphoma cells.

FIG. 13B illustrates the effect of dexamethasone alone and in combination with paroxetine, sertraline, and an exemplary compound of the present invention on viability of human Jurkat lymphoma cells.

FIG. 14 illustrates the effect of setraline, paroxetine, and exemplary compounds of the present invention on cell viability and proliferation in CEM/C1 cell-lines.

FIG. 15 illustrates the effect of doxorubicin (1-10 μM) and exemplary compounds of the present invention on cell proliferation in CEM/C1 human leukemia cells.

FIG. 16 illustrates the effect of several compounds, including an exemplary compound of the present invention, on tumor volume in mice.

FIG. 17 illustrates the effect of several compounds, including an exemplary compound of the present invention, on tumor weight in mice.

FIG. 18 illustrates the effect of several compounds, including an exemplary compound of the present invention, on the body weight of mice.

### DETAILED DESCRIPTION OF THE INVENTION

The present invention provides a compound of the formula A-L-B (I), wherein A is represented by the formulae (A1), (A2), or (A3):

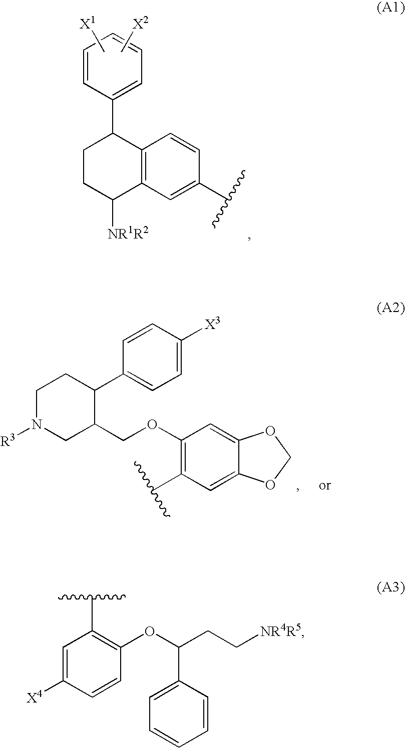

wherein:
R¹, R², R³, R⁴ and R⁵ are the same or different and each is independently a hydrogen or alkyl,
X¹ and X² are the same or different and each is independently a hydrogen, a halogen, haloalkyl, alkoxy, or a cyano,
X³ is a hydrogen, alkyl, alkoxy, haloalkyl, a hydroxyl, halogen, alkylthio, or an arylalkoxy, and
   X⁴ is a halogen, haloalkyl, alkyl, alkoxy, or alkenyl; L is a linking group comprising two carbon atoms; and B is an alkyl, alkenyl, alkynyl or aralkyl comprising at least one substituent of the formula Q, wherein the alkyl, alkenyl, alkynyl or aralkyl is optionally substituted with one or more halogens, hydroxyl, cyano, nitro, amino, or thiol:Q is OR⁶, OC(O)R⁶, C(O)R⁶, C(S)R⁶, CO₂R⁶, C(O)SR⁶, C(O)NR⁶R⁷, C(S)NR⁶R⁷, NR⁶R⁷, NR⁶C(O)R⁷, NR⁶C(S)R⁷, NR⁶C(O)NR⁷R⁸, NR⁶C(S)NR⁷R⁸, NR⁶SO₂R⁷, NR⁶SO₂NR⁷R⁸, SR⁶, SC(O)R⁶, SC(O)NR⁶R⁷, S(O)R⁶, SO₂R⁶, SO₂NR⁶R⁷, or NR⁶SO₂NR⁷R⁸, wherein R⁶, R⁷, and R⁸ are the same or different and each is independently a hydrogen, a C₁₋₆ alkyl, an aryl, an aralkyl, or a pharmaceutically acceptable solubility modifying group; or a salt, ester, or prodrug thereof, which may include, e.g., a pharmaceutically acceptable salt, ester, etc.

In one embodiment, B is —(CH₂)ₙ(CHR⁹)ₘQ, —Ar(CH₂)ₙ(CHR⁹)ₘQ, —(CH₂)ₙAr(CHR⁹)ₘQ or —(CH₂)ₙ(CHR⁹)ₘArQ, wherein m and n are the same or different and each is independently an integer of from 0 to about 6, provided that .m and n are not both zero when B is —(CH₂)ₙ(CHR⁹)ₘQ; Ar is a bivalent aryl (which is covalently bonded to the L and the (CH₂)ₙ, e.g., in —Ar(CH₂)ₙ(CHR⁹)ₘQ, covalently bonded to the —(CH₂)ₙ and the (CHR⁹)ₘ in —(CH₂)ₙAr(CHR⁹)ₘQ, covalently bonded to the (CHR⁹)ₙ, and the Q in —(CH₂)ₙ(CHR⁹)ₘArQ, etc.); R⁹ is a hydrogen, a C₁₋₆ alkyl, or an aryl; and the Ar, (CH₂)ₙ and (CHR⁹)ₙ, are optionally substituted with one or more halogens, hydroxyl, cyano, nitro, amino, or thiol. For example, B can be —(CH₂)ₙQ, —(CH₂)ₙArQ or —Ar(CH₂)ₙQ, wherein n is from 0 to about 6 provided that n is not zero when B is —(CH₂)ₙQ. In a preferred embodiment, B is —(CH₂)ₙQ, n is about 3, and/or Q is OR⁶, OC(O)R⁶, NR⁶R⁷, SR⁶ or SC(O)R⁶.

L can include any suitable linking group comprising two carbon atoms. For example, L can include a linker (e.g., a two-carbon linker) comprising carbon-carbon single bond, a linker (e.g., a two-carbon linker) comprising a carbon-carbon double bond, or a linker (e.g., a two-carbon linker) comprising a carbon-carbon triple bond. In one embodiment, L is a carbon-carbon triple bond. In some embodiments, L is a carbon-carbon friple bond, and R⁶, R⁷, and R⁸ are hydrogen. In other embodiments, L is a carbon-carbon triple bond, and n is from 2 to 4.

As utilized herein, the term “alkyl” generally includes straight-chain and branched-chain alkyl radicals, preferably containing from 1 to about 10 carbon atoms, e.g., from about 1 to about 8 carbon atoms, e.g., from about 1 to about 6 carbon atoms. Examples of alkyl substituents include methyl, ethyl, propyl, isopropyl, n-butyl, sec-butyl, isobutyl, tert-butyl, pentyl, isoamyl, hexyl, and the like.

The term “alkenyl” generally includes straight-chain and branched-chain alkenyl radicals having one or more carbon-carbon double bonds and preferably containing from 2 to about 10 carbon atoms, e.g., from 2 to about 8 carbon atoms, e.g., from 2 to about 6 carbon atoms. Examples of alkenyl substituents include vinyl, allyl, 1,4-butadienyl, isopropenyl, and the like.

The term “alkynyl” generally includes straight-chain and branched-chain alkynyl radicals having one or more carbon-carbon triple bonds and preferably containing from 2 to about 10 carbon atoms, e.g., from 2 to about 8 carbon atoms, e.g., from 2 to about 6 carbon atoms. Examples of alkynyl substituents include ethynyl, propynyl (propargyl), butynyl, and the like.

The term “alkoxy” generally includes alkyl ether radicals, wherein the term “alkyl” is as defined herein. Examples of alkoxy radicals include C₁₋₁₀ alkoxy radicals, C₁₋₈ alkoxy radicals, and C₁₋₆ alkoxy radicals. Specific examples of alkoxy radicals include methoxy, ethoxy, n-propoxy, isopropoxy, n-butoxy, isobutoxy, sec-butoxy, tert-butoxy, hexanoxy, and the like.

The term “alkylthio” generally includes alkyl thioether radicals, wherein the term “alkyl” is as defined herein. Examples of alkylthio radicals include C₁₋₁₀ alkthio radicals, C₁₋₈ alkylthio radicals, and C₁₋₆ alkthio radicals. Specific examples of alkylthio radicals include methylthio (SCH₃), ethylthio (SCH₂CH₃), n-propylthio, isopropylthio, n-butylthio, isobutylthio, sec-butylthio, tert-butylthio, n-hexylthio, and the like.

The term “aryl” refers to an aromatic carbocyclic radical, as commonly understood in the art, and includes monocyclic and polycyclic aromatics such as, for example, phenyl and naphthyl radicals.

The term “aralkyl” refers to an alkyl, as defined herein, substituted with one or more aryl moieties as defined herein. Preferably, the alkyl portion of the aralkyl is a C₁₋₁₀ alkyl, e.g. a C₁₋₈ alkyl, e.g., a C₁₋₆alkyl, wherein at least one hydrogen atom of the C₁₋₆ alkyl moiety is replaced by at least one aryl substituent. Examples thereof include benzyl, 1-phenethyl, 2-phenethyl, 3-phenylpropyl, 2-phenyl-1-propyl, and the like.

The term “arylalkoxy” generally includes alkoxy substituents, as defined herein, substituted with one or more aryls as defined herein. Examples of aralkoxy substituents include aryl(C₁₋₁₀)alkoxy, aryl(C₁₋₈)alkoxy, and aryl(C₁₋₆)alkoxy. Specific examples include phenylmethoxy, 2-phenylethoxy, 2-phenyl-1-propoxy, and the like.

The term “haloalkyl” generally includes alkyl substituents, as defined herein, substituted with one or more halogen atoms. Preferably, the alkyl portion of the haloalkyl is a C₁₋₁₀ alkyl, e.g., a C₁₋₈ alkyl, e.g., a C₁₋₆ alkyl. Examples of haloalkyl radicals include: C₁₋₆ fluorinated alkyl, such as trifluoromethyl, pentafluoroethyl, 2-fluoroethyl, 2-fluoro-1-propyl, and the like; C₁₋₆ chlorinated alkyl, such as chloromethyl, 2-chloroethyl, 2-chloro-1-propyl, and the like; C₁₋₆ brominated alkyl, such as bromomethyl, 2-bromoethyl, 2-bromo-1-propyl, and the like; C₁₋₆ iodinated alkyl, such as iodomethyl, 2-iodoethyl, 2-iodo-1-propyl, and the like.

The term “solubility modifying group” generally includes substituents that are useful in the art for modifying the solubility of a compound. It will be appreciated that the “solubility modifying group” can alter the molecular weight and lipophilicity of a particular compound, which, in turn, can impact the bioavailability of a particular compound, modulate or control tissue distribution, modify the ability of a particular compound to penetrate the blood-brain barrier, facilitate penetration into the skin for topical applications and/or facilitate systemic administration by a transdermal administration, and the like. The “solubility modifying group” can be charged or neutral and can be lipophilic or hydrophilic. Exemplary “solubility modifying groups” include, polyalcohols (e.g., polyethylene glycol having from about 2 to 25 units), polyol ethers, copolymers of ethylene and propylene glycol, esters of polyethylene glycols (e.g., laurate esters of polyethylene glycols), triphenylmethyl, naphthylphenylmethyl, palmitate, distearylglyceride, didodecylphosphatidyl, cholesteryl, arachidonyl, octadecanyloxy, tetradecylthio, alkyl groups, aryl groups, heteroaryl groups, hydroxyacids (e.g., lactic acid), amino acids, and the like. It will be appreciated that the solubility modifying group (and other substituents on the molecule) can be employed to inhibit (or even prevent) transport of the molecule across the blood-brain barrier, e.g., to minimize any psychotropic side effects that may be associated with the parent molecule.

Prodrugs of the compound of the present invention can include derivatives or analogs of the type that are understood in the art to be useful as prodrugs of biologically active compounds. The prodrugs may be active or inactive and, by virtue of chemical or enzymatic attack, can be converted to the parent drug in vivo before or after reaching a particular site of action. Prodrugs can include derivatives such as, e.g., esters and the like, which can be prepared, e.g., by reacting the active compound with a suitable acylating agent if the active compound includes a suitably reactive alcohol functional group. Prodrugs also can include carrier-linked prodrugs, bioprecursors, and the like. A carrier-linked prodrug, for example, can result from a temporary linkage of the active molecule with a transport moiety. Such prodrugs typically are less active or inactive relative to the parent active drug. The transport moiety can be chosen for its non-toxicity and its ability to ensure the efficient release of the active principle. A bioprecursor can result from a molecular modification of the active drug itself, e.g., by generation of a new molecule that is capable of acting as a substrate for one or more metabolizing enzymes whereby the action of a metabolizing enzyme produces the active drug in vivo. See also: WO 2006/0046967.

It will be appreciated that prodrugs can be employed to alter a variety of properties, including drug pharmacokinetics, stability, solubility, toxicity, specificity, duration of the pharmacological effect of the drug, and the like. By altering pharmacokinetics, the drug bioavailability can be increased, e.g., by increasing absorption, modulating distribution (e.g., systemically or in one or more particular tissues), controlling biotransformation, controlling the rate excretion of the drug, reducing acute toxicity, and the like. It is well within the skill of an ordinarily skilled artisan to design an develop a suitable prodrug of a particular biologically active molecule. In designing such prodrugs, factors taken into consideration can include, for example, the type of linkage that exists between the carrier and the drug (typically a covalent bond), the biological activity or toxicity of the prodrug relative to the active principle, the cost of preparing the prodrug, ease of synthesis, the reversibility of conversion to the active principle, and the like. Prodrugs may be prepared, e.g., by forming an ester, hemiester, carbonate ester, nitrate ester, amide, hydroxamic acid, carbamate, imine, mannich base, enamine, and the like. Prodrugs also may be prepared by functionalizing an active agent with an azo, a glycoside, a peptide, an ether, and the like, or by forming a salt, a complex, a phosphoramide, an acetal, a hemiacetal, a ketal, and the like.

In one embodiment, the compound of the present invention is of the formula A-L-B (I), wherein A is represented by formula (A1), and X¹ and X² are the same or different and each is independently a halogen. Alternatively or additionally, when A is of the formula (A1), R¹ and R² can be the same or different whereimeach is independently a hydrogen or a methyl, e.g., wherein one of R¹ and R² is a methyl and the other is a hydrogen, or wherein both R¹ and R² are hydrogen or methyl. An exemplary substituent of the formula A1 is represented by formula:

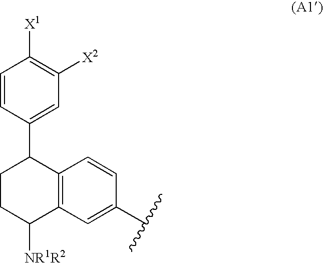

wherein X¹ and X² are the same or different and each is halogen, and R¹ and R² are the same or different and each is independently a hydrogen or a methyl. When A is of the formula (A1′), X¹ and X² preferably are chlorine, and one of R¹ and R² is a hydrogen and the other is a methyl.

In another embodiment, the compound of the present invention is of the formula A-L-B (I), wherein A is represented by formula (A2). When A is of the formula (A2), R³ preferably is hydrogen. Alternatively or additionally, when A is of the formula (A2), X³ preferably is a hydrogen or a halogen, and is more preferably a halogen (e.g., fluorine). In one series, when A is represented by formula (A2), R³ is a hydrogen and X³ is a halogen (which is preferably fluorine).

In another embodiment, the compound of the present invention is of the formula A-L-B (I), wherein A is represented by formula (A3). When A is of the formula (A3), R⁴ and R⁵ are the same or different and each can be, e.g., independently a methyl or a hydrogen. For instance, when A is of the formula (A3), one of R⁴ and R⁵ can be a methyl and the other can be a hydrogen. Alternatively or additionally, when A is of the formula (A3), X⁴ can be a C₁₋₆ haloalkyl, such as for example, C₁₋₆ fluorinated alkyl (e.g., trifluoromethyl). In one series, when A is of the formula (A3), one of R⁴ and R⁵ is methyl and the other is hydrogen, and X⁴ is trifluoromethyl.

It will be appreciated that the compound of the formula A-L-B (I) includes geometrical and optical isomers, e.g., diasteomers and diastereomeric mixtures, enantiomers (e.g., a substantially pure enantiomer or an enantiomeric mixture), and molecules of the same general formula having any other suitable combination of chiral centers. For instance, A can include substituents of the formulae:

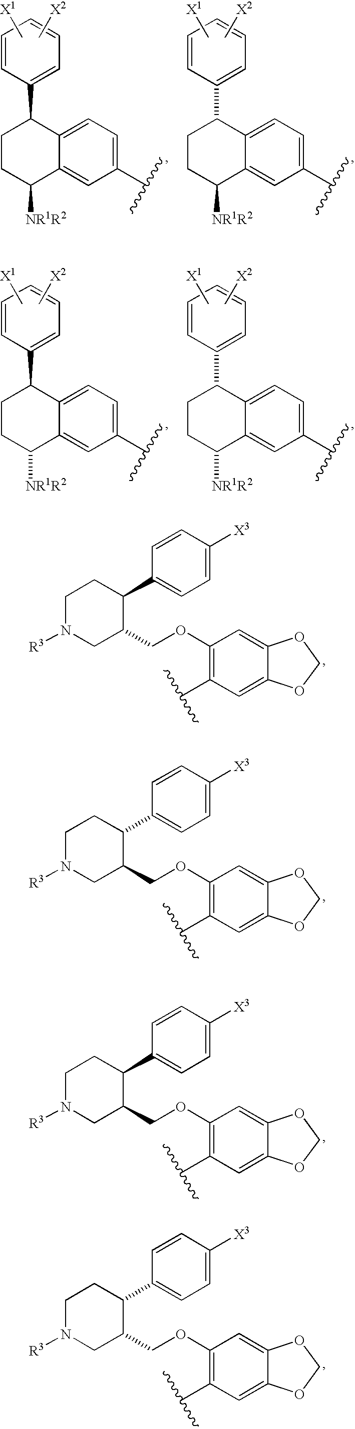

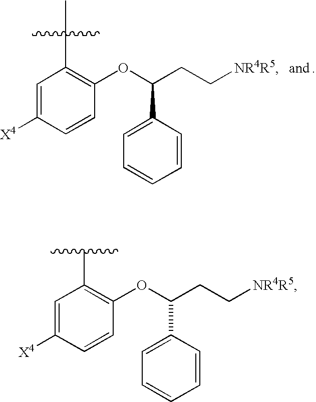

and combinations thereof, wherein X¹-X³ and R¹-R⁵ are as define herein.

The compound of the formula A-L-B (I) also includes, e.g., solvates, hydrates and polymorphs.

Exemplary compounds of the present invention include the following:

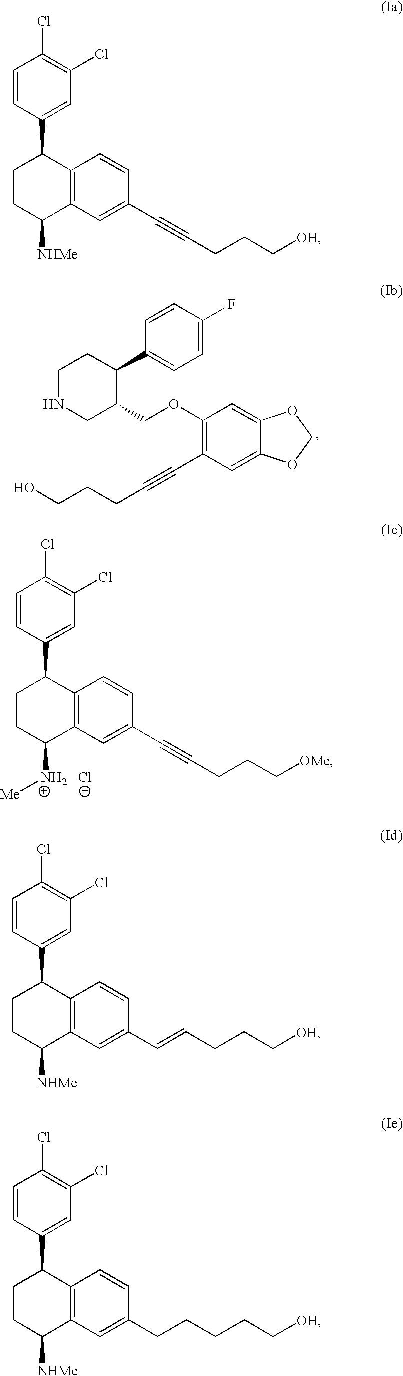

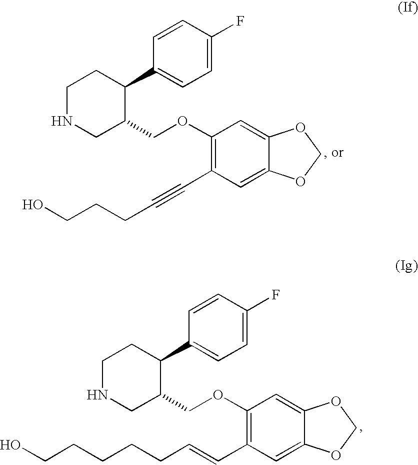

and pharmaceutically acceptable salts, esters, and prodrugs thereof.

The present invention further provides a pharmaceutical composition comprising a pharmaceutically acceptable carrier and a therapeutically effective amount of at least one compound of present invention. The therapeutically effective amount preferably is an antiproliferative effective amount, which can include, for example, an amount of one or more compounds of the present invention required to therapeutically inhibit abnormal or undesirable cellular proliferation in a particular patient, e.g., an anti-cancer effective amount. The therapeutically effective amount preferably includes the dose necessary to achieve an “effective level” of one or more of the active compounds in an individual patient. The effective level can be defined, for example, as the amount required in an individual patient to achieve an antiproliferative effective blood and/or tissue level of a compound of the present invention, or the level that is effective to kill or inhibit the growth (e.g., suppress, retard or decrease the growth rate) of cells associated with a particular proliferative disease or disorder (i.e., diseases associated with abnormal or undesirable cell proliferation) in the patient. The effective level also may be chosen, for example, as the blood or tissue level that corresponds to a concentration of a compound of the present invention effective to kill or inhibit the growth of cells associated with proliferative diseases or disorders, e.g., based on an assay, which is reasonably predictive of clinical efficacy. The effective level also may be chosen, for example, as the blood level required to kill or inhibit the growth of cancer cells, e.g., tumor cells, based on a screening assay that is reasonably predictive of clinical efficacy.

The effective level also can be defined, for example, as the concentration of one or more compounds of the present invention needed to inhibit markers of the proliferative disease or disorder in the patient's blood, or which can be shown to slow or stop the growth of the cells associated with the patient's proliferative disorder, or which causes the patient's proliferative disease or disorder to regress or disappear, or which alleviates one or more symptoms associated with the disease or disorder, or renders the patient asymptomatic to the particular proliferative disease or disorder, or which renders an improvement in the patient's subjective sense of condition.

One skilled in the art can easily determine the appropriate dose, schedule, and method of administration for the exact formulation or composition being used in order to achieve the desired effective level in the patient. One skilled in the art also can readily determine by a direct (e.g., analytical chemistry) and/or indirect (e.g., with clinical chemistry indicators) analysis of appropriate patient samples (e.g., blood and/or tissues), or, in the case of cancer, e.g., by direct or indirect observations of the shrinkage or inhibition of growth of the individual patient's tumor. The effective level may be achieved, for example, by administering one or more compounds of the present invention in an amount effective to ameliorate undesired symptoms associated with the proliferative disease or disorder, prevent the manifestation of such symptoms before they occur, slow the progression of the proliferative disease or disorder, slow the progression of symptoms associated with the proliferative disease or disorder, initiate the onset of a remission period, slow any irreversible damage that is caused in a progressive chronic stage of the disease or disorder, delay the onset of the progressive chronic stage of the disease or disorder, reduce the severity of the disease or disorder, cure the disease or disorder, improve the survival rate of patients suffering from the disease or disorder, initiate a more rapid recovery from the disease or disorder, kill the cells associated with the proliferative disease or disorder, inhibit the cell proliferation associated with the proliferative disease or disorder, and/or prevent (e.g., decrease the likelihood of) the disease form occurring.

There are many references in the art that describe how to determine the protocols of administering anti-proliferative agents to patients. See e.g., “Cancer Chemotherapy: Principles and Practice” ed., Chabner and Collins, J. B. Lippincott, 1990, especially chapter 2, by J. B. Collins. It will be appreciated that the actual dose and schedule for drug administration for each patient can vary depending on interindividual differences in pharmacokinetics, drug disposition, bioavailability, metabolism, and the like. It will also be appreciated that the effective level of a particular compound of the present invention can vary when a compound of the present invention is used alone or in combination with or more anti-proliferative compounds other than a compound of the present invention.

The method of the present invention for treating a proliferative disease or disorder may be made more effective by administering one or more known anti-proliferative drugs in combination with one or more compounds of the present invention (e.g., during the course of therapy, e.g., by co-administration). Known anti-proliferative compounds can include one or more compounds approved for marketing in the United States and those that will become approved in the future. See, e.g., Introduction to Cancer Therapy (J. E. Niederhuber, ed.), Chapter 2, by B. C. Decker, Inc., Philadelphia, 1993, pp. 11-22. In the case of cancer, such other anti-proliferative compounds may include, e.g., doxorubicin, bleomycin, vincristine, vinblastine, VP-16, VW-26, cisplatin, procarbazine, and taxol for solid tumors in general; alkylating agents, such as BCNU, CCNU, methyl-CCNU and DTIC, for brain or kidney cancers; and antimetabolites such as 5-FU and methotrexate (e.g., for colon cancer).

In accordance with the present invention, the dose administered to a patient preferably is sufficient to produce an effective level in the patient over a reasonable time frame. It will be appreciated that the amount of active ingredient that can be combined with the carrier materials to produce a single dosage form will vary depending upon the patient treated and the particular mode of administration. One skilled in the art will recognize that the specific dosage level for any particular patient will depend upon a variety of factors including, for example, the activity of the specific compound employed, age, body weight, general health, sex, diet, time of administration, route of administration, rate of excretion, drug combination and severity of the particular proliferative disease or disorder being treated. The size of the dose will also be determined by the existence, nature, and extent of any adverse side-effects that might accompany the administration of a particular compound. Other factors which affect the specific dosage can include, for example, bioavailability, metabolic profile, the pharmacodynamics associated with the particular compound to be administered in a particular patient, and the like.

One or more compounds of the present invention can be formulated into a pharmaceutical composition, e.g., by combining a therapeutically effective amount of one or more compounds of the present invention with a pharmaceutically acceptable carrier. Pharmaceutically acceptable carriers are well-known in the art and may include, e.g., pharmaceutical vehicles, adjuvants, excipients, diluents, and the like. Preferably, the pharmaceutically acceptable carrier is selected such that it is chemically inert with respect to the active agent(s). The pharmaceuatically acceptable carrier also is desirably selected such that it has minimal or no detrimental side effects or toxicity under the conditions of use. The choice of a carrier will be determined in part by the particular composition, as well as by the particular mode of administration.

One skilled in the art will appreciate that various routes of administering a drug are available and, although more than on route may be used to administer a particular drug, one particular route may provide a more immediate and more effective reaction than anther route. Furthermore, one skilled in the art will appreciate that the particular pharmaceutical carrier employed will depend, in part, upon the particular compound employed and the chosen route of administration.

The pharmaceutical composition of the present invention may be in a form suitable for oral administration, such as, for example, tablets, troches, aqueous or oily suspensions, dispersible powders or granules, emulsions, hard or soft capsules, syrups, solutions or elixirs. Compositions intended for oral use may be prepared according to any method known in the art for the manufacture of such pharmaceutical compositions, and such compositions can contain one or more agents including, for example; sweetening agents, flavoring agents, coloring agents, and preserving agents in order to provide a pharmaceutically elegant and/or palatable preparation. Tablets can contain the active ingredient in admixture with non-toxic pharmaceutically acceptable excipients which are suitable for the manufacture of tablets. Such excipients can include, for example, inert diluents such as, for example, calcium carbonate, lactose, mannitol, calcium phosphate or sodium phosphate; granulating and disintegrating agents such as, for example; maize starch, corn starch, potato starch, and alginic acid; binding agents such as, for example, starch, gelatine or acacia, lubricating agents such as, for example, stearic acid or talc, and the like. Such excipients can also include microcrystalline cellulose, colloidal silicon dioxide, croscarmellose, and the like. The tablets may also include other excipients, colorants, diluents, buffering agents, moistening agents, preservatives, flavoring agents, and pharmacologically compatible carriers. The tablets may be uncoated, or they may be coated by known techniques to delay disintegration and absorption in the gastrointestinal tract and thereby provide a sustained action over a longer period. A time delay material, for example, glyceryl monostearate or glyceryl distearate, alone or with a wax, may also be employed. Formulations for oral use also can be presented as hard gelatin capsules, wherein the active ingredient is mixed with an inert solid diluent, for example calcium carbonate, calcium phosphate or kaolin, or as soft gelatin capsules wherein the active ingredient is mixed with water or an oil medium, for example arachis oil, peanut oil, liquid paraffin or olive oil.

Furthermore, formulations suitable for oral administration may include liquid solutions, which may consist of an effective amount of one or more compounds of the present invention dissolved or dispersed in one or more diluents, such as, e.g., water, saline, or orange juice; capsules, sachets or tablets, each containing a predetermined amount of the active ingredient as solids orgranules; solutions orsuspensionsin an aqueous liquid; and oil-in-water emulsions or water-in-oil emulsions. Aqueous suspensions, for example, can contain the active material(s) in admixture with excipients suitable for the manufacture of aqueous suspensions. Such excipients include suspending agents, for example, sodium carboxymethyl cellulose, methylcellulose, hydroxypropylmethylcellulose, sodium alginate, polyvinylpyrrolidone, gum tragacanth and gam acacia. Dispersing or wetting agents may include natural-occurring phosphatides, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol, for example, polyoxyethylene sorbitol monooleate, or condensation products of ethylene oxide with partial esters derived from fatty acids and hexitol anhydrides, for example, polyoxyethylene sorbitan mono-oleate. The aqueous suspensions also can contain one or more preservatives, for example, ethyl or n-propyl p-hydroxy benzoate, one or more coloring agents, one or more flavoring agents and one or more sweetening agents such as, for example, sucrose or saccharin.

Formulations suitable for oral administration also can include lozenges comprising the active ingredient in a flavor, e.g., sucrose and acacia or tragacanth; pastilles comprising the active ingredient in an inert base, such as, e.g., gelatin and glycerin, or sucrose and acacia; and mouthwashes comprising the active ingredient in a suitable liquid carier; as well as creams, emulsions, gels, and the like containing a therapeutically effective amount of the active ingredient(s).

Oily suspensions may be formulated by suspending the active ingredient in a vegetable oil, for example arachis oil, olive oil, sesame oil or coconut oil, or in a mineral oil such as liquid paraffin. The oil suspensions may contain a thickening agent, for example beeswax, hard paraffin or cetyl alcohol. Sweetening agents, such as those set forth above, and flavoring agents may be added to provide a palatable oral preparation. If desired, such compositions may be preserved by the addition of an antioxidant such as, for example, ascorbic acid, or an antimicrobial agent.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, also may be present.

The pharmaceutical composition of the present invention also can be in the form of an oil-in-water emulsion. The oily phase can be a vegetable oil, for example, olive oil or arachis oils, or a mineral oil, for example liquid paraffin or mixtures of these. Suitable emulsifying agents may include naturally-occurring gums, for example gum acacia or gum tragacanth, naturally-occurring phosphatides, for example soya bean lecithin, and esters or partial esters derived from fatty acids and hexitol anhydrides, for example sorbitan mono-oleate, and condensation products of the said partial esters and ethylene oxide, for example polyoxyethylene sorbitan mono-oleate. The emulsions also can contain sweetening and flavoring agents.

The pharmaceutical composition can be in the form of a sterile injectable preparation, for example, as a sterile injectable aqueous or oleagenous suspension. Suitable suspensions for parenteral administration can be formulated according to the known art using those suitable dispersing or wetting agents and suspending agents which have been mentioned above. Formulations suitable for parenteral administration also can include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The sterile injectable preparation can be in the form of a solution or a suspension in a non-toxic parenterally-acceptable diluent or solvent, for example, as a solution in water or 1,3-butanediol. Among the acceptable vehicles and solvents that can be employed, for example, are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as, for example, oleic acid find use in the preparation of injectables.

The compound(s) or pharmaceutical composition(s) of the present invention also can be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include, for example, cocoa butter and polyethylene glycols.

Formulations suitable for vaginal administration may be presented as pessaries, tampons, creams, gels, pastes, foams, or spray formulas containing, in addition to the active ingredient, such carriers as are known in the art to be appropriate. Similarly, the active ingredient may be combined with a lubricant as a coating on a condom.

Formulations suitable for topical administration may be presented as creams, gels, pastes, or foams, containing, in addition to the active ingredient, such carriers as, are known in the art to be appropriate.

The compound(s) or pharmaceutical composition(s) of the present invention also, can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non-pressured preparations such as in a nebulizer or an atomizer.

The formulations can be presented in unit-dose or multi-dose sealed containers, such as ampules and vials, and can be stored in a freeze-dried (lyophilized) condition requiring only the addition of the sterile liquid excipient, for example, water, for injection, immediately prior to use. Extemporaneous injection solutions and suspensions can be prepared from sterile powders, granules, and tablets of the kind previously described.

A variety of methods known in the art, including those discussed herein, can be employed to administer the compound(s) or the composition(s) of the present invention. In one embodiment, the compound(s) or composition(s) of the present invention can be administered in an amount effective to treat abnormal or undesirable cell proliferation in a patient, e.g., an amount effective to treat cancer, e.g., an anti-cancer effective amount, e.g., an amount of one or more compounds or compositions of the present invention needed to inhibit the proliferation of cancer cells in a patient as described herein. The compound(s) or composition(s) of the present invention can be administered, e.g., intravenously, orally, parenterally, or topically as described herein.

Moreover, the present invention provides a method of treating a disease or disorder associated with abnormal or undesirable cell proliferation in a patient, which method includes administering to the patient a therapeutically effective amount of at least one compound or pharmaceutical composition of the present invention. The present invention also provides a method of treating cancer in a patient, which method includes administering to the patient a therapeutically effective amount of at least one compound or pharmaceutical composition of the present invention. The method of treating cancer in accordance with the present invention can be applied toward the treatment of multidrug resistant cancer.

The methods of the present invention also may be used for treating skin diseases or disorders, proliferative diseases or disorders, multidrug resistant proliferative diseases or disorders, non-malignant proliferative diseases or disorders, or diseases or disorders associated with proliferation of cells of the immune system. Diseases or disorders associated with proliferation of cells of the immune system include, for example, rosacea, acne vulgaris, and seborrheic dermatitis. The compounds of the present invention also may be administered, e.g., in accordance with the methods described herein, for treating corticosteroid-resistant hematological cancer.

Non-malignant proliferative diseases or disorders, which may be treated in accordance with the present invention, can include, for example, psoriasis, psoriasis hyperkeratosis, scleroderma, actinic keratosis, eczema, and diseases or disorders associated with the proliferation of cells of the immune system, e.g., multiple sclerosis, Crohn's disease, ulcerative colitis, rheumatoid arthritis, systemic lupus erythematosis, inflammatory bowel disorder, atopic dermatitis, and contact dermatitis. Malignant proliferative diseases or disorders, which may be treated in accordance with the present invention, can include, for example, prostate cancer, leukemia, lymphoma, skin cancer, brain cancer, colon cancer, lung cancer, breast cancer, basal cell carcinoma, and melanoma.

The present invention also provides a method of treating a disease or disorder associated with abnormal or undesirable cell proliferation in a patient which method includes administering to the patient a therapeutically effective amount of at least one compound or composition of the present invention and a therapeutically effective amount of at least one additional chemotherapeutic agent (e.g., an anti-cancer agent such as, e.g., methotrexate, anti-tyrosine kinase agents, e.g., Gleevec, and the like, and combinations thereof) other than a compound of the present invention. Preferably, the additional chemotherapeutic agent produces an additive or synergistic antiproliferative effect when administered in combination with one or more compounds of the present invention. In one embodiment, the method comprises administering to the patient a therapeutically effective amount of at least one compound of the present invention and a therapeutically effective amount of a corticosteroid (e.g., an antiproliferative or anti-inflammatory effective amount of one or more corticosteroids), wherein the corticosteroid produces an additive or synergistic antiproliferative or anti-inflammatory effect when administered in combination with one or more compounds of the present invention.

The present invention also provides a method of treating cancer, which method includes administering to a patient a therapeutically effective amount of at least one compound or pharmaceutical composition of the present invention and a therapeutically effective amount of at least one additional chemotherapeutic agent (e.g., an anti-cancer agent) other than a compound of the present invention. The additional chemotherapeutic agent preferably produces an additive or synergistic antiproliferative effect when administered in combination with one or more compounds of the present invention.

The present invention also provides processes for preparing compounds of the present invention. An exemplary process of the present invention includes reacting a compound of the formula:

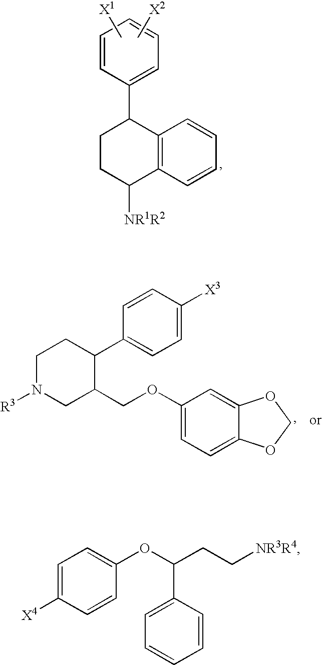

with a halogenating agent to produce a halogenated compound of the formula:

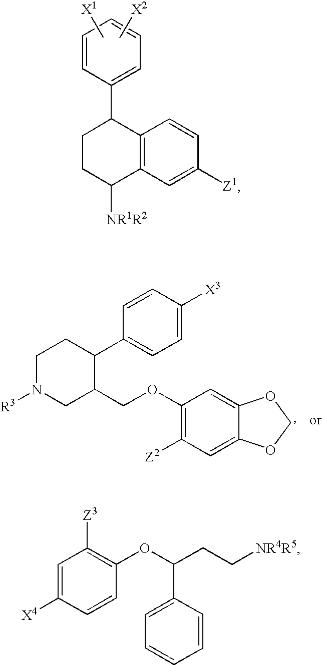

wherein, Z¹, Z², and Z³ are the same or different and each is independently a halogen, and R¹-R⁵ and X¹-X⁴ are as defined herein; coupling the halogenated compound with a compound of the formula L-B, wherein L is a linking group comprising a carbon-carbon triple bond, which is preferably a terminal acetylene (HC≡C—), and B is as defined herein (e.g., alkyl, alkenyl, alkynyl, aralkyl, —(CH₂)ₙ(CHR⁹)ₘQ, —Ar(CH₂)ₙ(CHR⁹)ₘQ, —(CH₂)ₙAr(CHR⁹)ₘQ or —(CH₂)ₙ(CHR⁹)ₘArQ), wherein R¹-R⁹, X¹-X⁴, Q, m and n are as defined herein, to produce a coupling product that includes a carbon-carbon triple bond; optionally converting the carbon-carbon triple bond in the coupling product into a carbon-carbon double bond or carbon-carbon single bond; optionally introducing a pharmaceutically acceptable solubility modifying group to the coupling product; and optionally converting the coupling product into a pharmaceutically acceptable salt, ester, or prodrug, to produce a compound of the formula A-L-B (I) as defined herein.

The halogenating agent can generally include any compound, reagent or combination of compounds and reagents, which is capable of halogenating (preferably by selectively introducing a halogen) to an aromatic ring. Exemplary halogenating agents may include, e.g., Br₂ (with or without a catalyst), Cl₂ (with or without a catalyst), I₂ (with or without a catalyst), N-chlorosuccinimide, N-bromosuccinimide, N-iodosuccinimide, and the like. Preferably, the halogenating agent used in the production process of the present innvention is N-chlorosuccinimide, N-bromosuccinimide, or N-iodosuccinimide.

The coupling reaction generally includes methods known in the art for introducing an alkyne substituent to a suitably reactive aromatic halide to produce the coupling product. For instance, the coupling process may be performed by way of a Sonogashira coupling reaction.

Methods for optionally converting the carbon-carbon triple bond into a carbon-carbon double bond or a carbon-carbon single bond are generally known in the art. For example, the carbon-carbon triple bond can be converted into a carbon-carbon double bond or a carbon-carbon single bond via reduction, (e.g., hydrogenation), hydroboration (and, optionally, further reacting the hydroborated intermediate, e.g., by oxidation), hydrohalogenation, halogenation, and the like.

Likewise, methods for optionally introducing a pharmaceutically acceptable solubility modifying group are known in the art. For example, when R⁷ is hydrogen, a pharmaceutically acceptable solubility modifying group may be introduced by esterifying a hydroxyl group with an acylating agent that includes a pharmaceutically acceptable solubility modifying group, or by alkylating a hydroxyl group with a pharmaceutically acceptable solubility modifying group.

In one embodiment, the process of the present invention utilizes the following compound as a starting material.

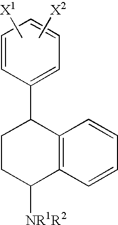

Such starting materials can be obtained using methods, which are well known in the art. See, e.g., U.S. Pat. No. 4,536,518, which describes methods of preparing sertraline and derivatives thereof. An exemplary process of the present invention is depicted in Scheme 1.

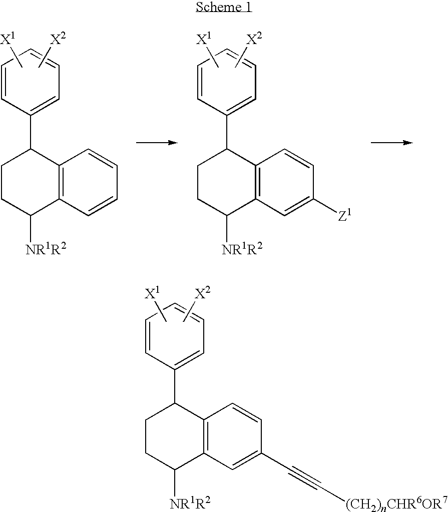

Another exemplary process of the present invention is depicted in Scheme 2.

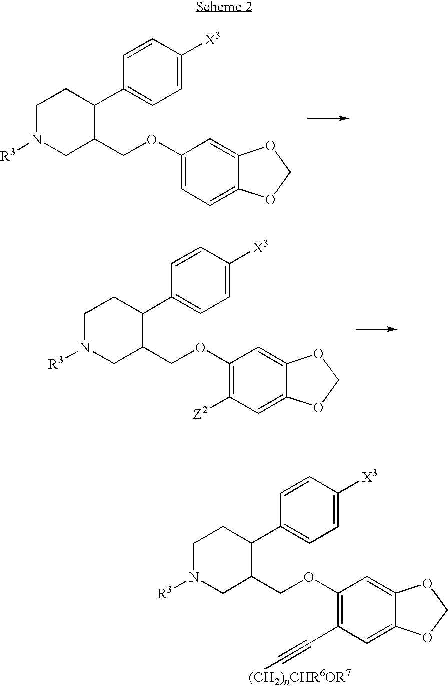

Starting materials for the process depicted in Scheme 2 can be obtained by methods, which are well known in the art. See, e.g., U.S. Pat. No. 4,007,196, which describes methods of preparing paroxetine and derivatives thereof. See also, e.g., U.S. Pat. No. 4,314,081, which describes methods of preparing fluoxetine and derivatives thereof, which can serve as intermediates for producing compounds of the formula A-L-B (I), wherein A is of the formula (A3), as defined herein.

Methods of preparing SSRI derivatives, which can serve as intermediates for producing compounds of the present invention, also are described in U.S. Pat. No. 5,320,825.

Another exemplary process of the present invention includes regioselectively formylating a compound of the formula:

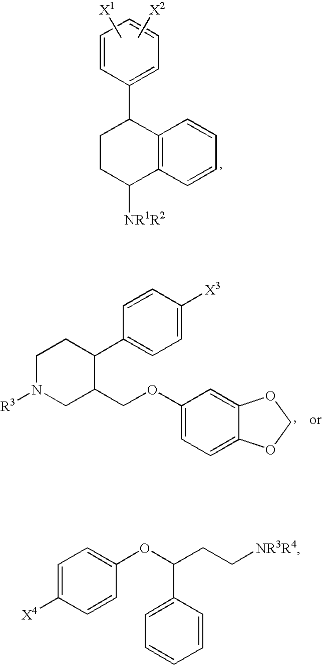

by reacting the compound with a formylating reagent (e.g., using suitable Vilsmeier-Haack conditions), to produce a formylated compound of the formula:

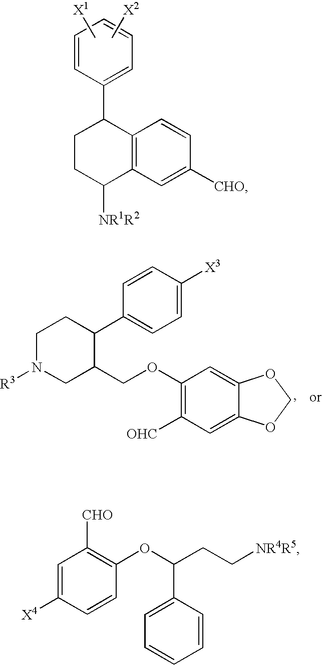

and reacting the formylated compound with a reagent capable of reacting with the formyl substituent (e.g., using a suitable a Wittig reagent or other aldehyde alkenylation reagent), to produce an alkenyl product of the formula:

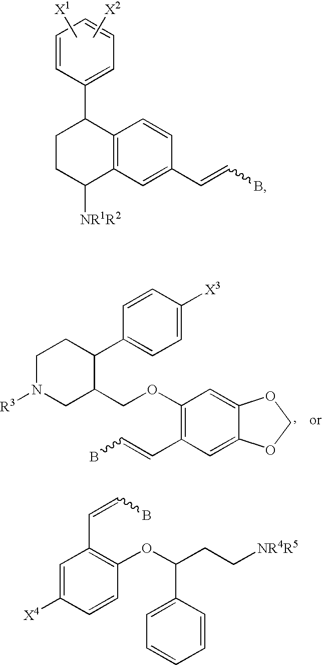

wherein B is as defined herein, and optionally converting the carbon-carbon double bond of the alkenyl product into a carbon-carbon single bond, optionally introducing a pharmaceutically acceptable solubility modifying group to the alkenyl product, and optionally converting the alkenyl product into a pharmaceutically acceptable salt, ester, or prodrug, to produce a compound of the formula A-L-B (I) as defined herein.

The formylating reagent can generally include any compound, reagent or combination of compounds and reagents, which is capable of formylating an aromatic ring. An exemplary formylating reagent, which can be used in the production process of the present invention, is the product of dimethyl formamide and POCl₃.

The reagent capable of reacting with the formyl substituent can include any compound, reagent or combination of compounds and reagents, which is capable of reacting with the formyl substituent. Exemplary reagents capable of reacting with the formyl substituent may include, e.g., Wittig reagents such as, for example, Ph₃P⁺CH₂B Br⁻, e.g., in the presence of a suitable base, wherein B is as defined herein.

The following examples further illustrate the invention but, of course, should not be construed as in any way limiting its scope.

### Example 1

This example demonstrates the synthesis of an exemplary compound of the present invention, (1S -cis)-4-(3,4-dichlorophenyl)-7-(5-hydroxy-1-pentyn-1-yl)-1,2,3,4-tetrahydro-N-methyl-1-naphthalenamine hydroiodide (Ia).

### (1S-cis)-4-(3,4-dichlorophenyl)-7-iodo-1,2,3,4-tetrahydro-N-methyl-1-naphthalenamine (1)

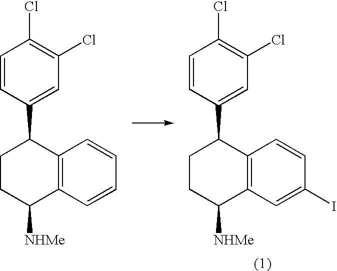

Trifluoromethanesulfonic acid (2.2 ml, 22 mmol) was added to a suspension of (1S-cis)-4-(3,4-dichlorophenyl)-1,2,3,4-tetrahydro-N-methyl-1-naphthalenamine hydrochloride (Sertraline hydrochloride) (2.5 g, 7.3 mmol) in 8 ml dichloromethane (DCM) and cooled to 0° C. under nitrogen. Following the complete dissolution of the salt, N-iodosuccinimide (1.3 g, 6.5 mmol) was added. The reaction was stirred for 17 h and a second portion of N-iodosuccinimide (0.3g, 1.5 mmol) was added. After 24 h, a 2 N aqueous sodium hydroxide solution (15 mL) was added slowly and the resulting mixture was extracted three times with 15 mL of diethyl ether. The combined organic extracts were washed with a saturated aqueous solution of sodium thiosulfate (15 mL) and then brine (15 mL), and dried over MgSO₄. The ether was evaporated under reduced pressure and the crude yellow oil was purified on silica gel (eluent:ethyl acetate:hexanes, 0:100 to 50:50) to yield 1.0 g (35%) of the compound represented by formula (1) as a dark yellow oil.

### (1S-cis)-4-(3,4-dichlorophenyl)-7-(5-hydroxy-1-pentyn-1-yl)-1,2,3,4-tetrahydro-N-methyl-1-naphthalenamine hydroiodide (Ia)

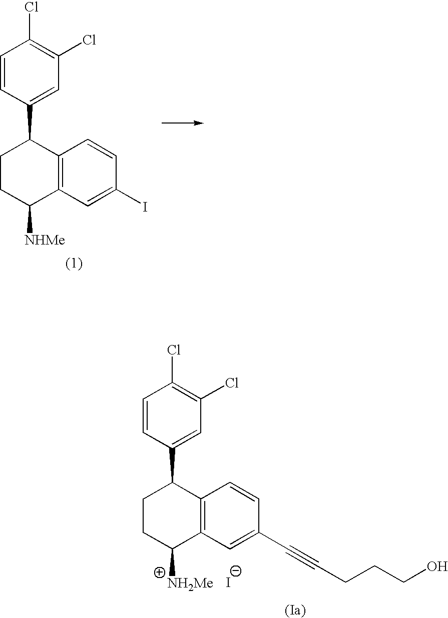

The compound represented by formula (1) (250 mg, 0.58 mmol), Pd(PPh₃)₂Cl₂ (9 mg, 0.013 mmol), CuI (5 mg, 0.025 mmol), 4-pentyn-1-ol (50 mg, 0.64 mmol), diethylamine (0.97 ml), and DMF (1.5 ml) were mixed and stirred under nitrogen overnight. The solvent was removed under reduced pressure and the residue was purified on silica gel (hexanes:ethyl acetate, 50:50 to 0:100) to yield the compound represented by formula (Ia) (70%) as an off-white solid. ¹H NMR (400 MHz, CDCl₃/CD₃OD (2%)): δ 7.50 (br s, 1H), 7.21 (d, J=8.0 Hz, 1H), 7.14 (d, J=1.8 Hz, 1H), 7.06 (dd, J=8.0 Hz, J=1.3 Hz, 1H), 6.93 (dd, J=8.0 Hz, J=2.0 Hz, 1H), 6.63(d, J=8.0 Hz, 1H), 4.21 (br t, J=4.0 Hz, 1H), 3.84 (br t, J=6.8 Hz, 1H), 3.59 (t, J=6.4 Hz, 2H), 2.56 (s, 3H), 2.32 (t, J=6.8 Hz, 2H), 2.09 (m, 1H), 1.94 (m, 3H), 1.65 (quint, J=6.6 Hz, 2H). ¹³C NMR (100 MHz, CDCl₃/CD₃OD (2%)): δ 144.9, 138.0, 133.0, 132.3, 131.8, 131.4, 130.7, 130.6, 130.5, 130.2, 128.5, 122.8, 90.8, 80.0, 61.0, 56.4, 44.6, 31.1, 30.8, 27.7, 23.3, 15.7. HRMS (EI): calcd. for C₂₂H₂₃Cl₂NO (M⁺) 387.1157; found 387.1167.

### Example 2

This example illustrates the synthesis of an exemplary compound of the present invention, (3 S -trans)-3-((6-(5-hydroxy-1-pentyn-1-yl)-1,3-benzodioxol-5-yloxy)methyl)-4-(4-fluorophenyl)-piperidine (Ib). The synthesis is outlined in Scheme 3.

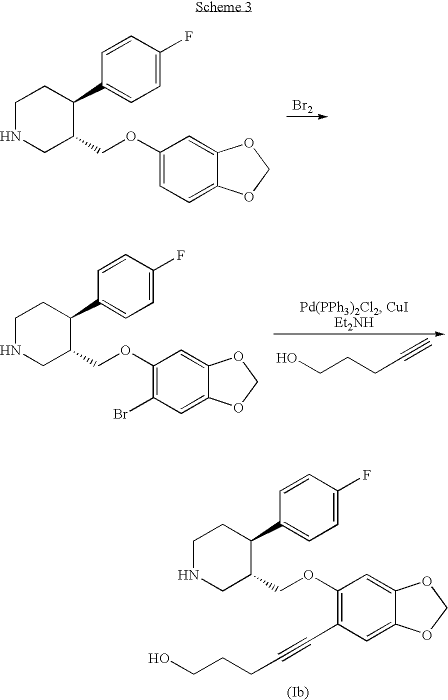

### (3S-trans)-3-((6-bromo-1,3-benzodioxol-5-yloxy)methyl)-4-(4-fluorophenyl)-piperidine

A solution of bromine (0.07 mL, 1.44 mmol) in 0.5 mL of dichloromethane was added dropwise to a suspension of (3S-trans)-3-((1,3-benzodioxol-5-yloxy)methyl)-4-(4-fluorophenyl)-piperidine hydrochloride (Paroxetine hydrochloride) (0.5 g, 1.37 mmol) in 4 mL of dichloromethane. After 2 h, 30 mL of water was added and the mixture was extracted with 20 mL of dichloromethane. The organic phase was washed twice with a saturated aqueous solution of sodium bicarbonate (2×30 mL), then with brine (30 mL) and finally with 30 mL of a saturated aqueous solution of sodium sulfite. The dichloromethane solution was dried over MgSO₄, and the solvent was evaporated under reduced pressure. The resulting crude brown oil was purified on silica gel (eluent methanol:ethyl acetate, 0:100 to 40:60) to yield 219 mg (39%) of yellow oil. ¹H NMR (200 MHz, CDCl₃): δ 7.22 (dd, J=8.7 Hz, J=5.5 Hz, 2H), 7.00 (t, J=8.7 Hz, 2H), 6.98 (s, 1H), 6.26 (s, 1H), 5.91 (s, 2H), 3.65 (dd, J=9.2 Hz, J=2.6 Hz, 1H), 3.49 (m, 2H), 3.28 (dm, J=11.2 Hz, 1H), 2.82 (m, 3H), 2.07 (m, 1H), 1.88 (m, 2H). ¹³C NMR (50 MHz, CDCl₃): δ 161.5 (d, J=243 Hz), 150.1, 147.5, 141.8, 139.4, 128.8 (d, J=7 Hz), 115.4 (d, J=21 Hz), 112.3, 101.9, 101.6, 96.7, 70.2, 49.6, 46.5, 43.7, 42.5, 34.4. MS (EI): calcd. for C₁₉H₁₉BrFNO₃ (M⁺) 407.1; found 407.1.

### (3S-trans)-3-((6-(5-hydroxy-1-pentyn-1-yl)-1,3-benzodioxol-5-yloxy)methyl)-4-(4-fluorophenyl)-piperidine (Ib)

(3S-trans)-3-((6-bromo-1,3-benzodioxol-5-yloxy)methyl)-4-(4-fluorophenyl)-piperidine (0.22 g, 0.54 mmol), Pd(PPh₃)₂Cl₂ (0.06 g, 0.08 mmol), CuI (0.01 g, 0.06 mmol), triphenylphosphine (0.014 g, 0.055 mmol), 4-pentyn-1-ol (0.06 mL, 0.6 mmol), triethylamine (1.4 mL, 10 mmol) and 5 mL of dry tetrahydrofuran (THF) were mixed and stirred in a pressure tube at 100° C. under nitrogen. After 3 days the solvent was removed under reduced pressure and the crude oil was dissolved in 15 mL of ethyl acetate and washed two times with a saturated aqueous solution of potassium carbonate and then with brine (15 mL each). The organic phase was dried over MgSO₄ and the solvent was evaporated under reduced pressure. The resulting crude brown oil was purified on silica gel (methanol:chloroform, gradient 0:100 to 20:80) to yield 10 mg (5%) of brownish oil. ¹H NMR (400 MHz, CDCl₃): δ 7.18 (dd, J=8.6 Hz, J=5.4 Hz, 2H), 6.99 (t, J=8.7 Hz, 2H), 6.74 (s, 1H), 6.19 (s, 1H), 5.87 (m, 2H), 3.97 (dt, J=10.6 Hz, J=6.8 Hz, 1H), 3.82 (dt, J=10.6 Hz, J=6.8 Hz, 1H), 3.71 (dd, J=12.0 Hz, J=2.8 Hz, 1H), 3.60 (dd, J=9.2 Hz, J=3.0 Hz, 1H), 3.48 (t, J=8.8 Hz, 1H), 3.23 (dm, J=12.1 Hz, 1H), 2.78 (dt, J=3.3 Hz, J=11.9 Hz, 1H), 2.73 (t, J=11.9 Hz, 1H), 2.55 (m, 3H), 2.25 (m, 1H), 1.85 (m, 4H). ¹³C NMR (100 MHz, CDCl₃): δ 161.5 (d, J=243 Hz), 155.6, 147.8, 140.9, 138.7, 128.6, 115.4 (d, J=21 Hz), 111.3, 101.2, 95.9, 92.1, 69.8, 61.1, 49.3, 45.9, 43.7, 42.1, 33.7, 31.5, 15.9. MS (FAB): calcd. for C₂₂H₂₄Cl₂NO (MH⁺) 412.1; found 412.1.

### Example 3

This example illustrates the synthesis of an exemplary compound of the present invention, (1S -cis)-4-(3,4-dichlorophenyl)-7-(5-methoxy-1-pentyn-1-yl)-1,2,3,4-tetrahydro-N-methyl-1-naphthalenamine hydrochloride (Ic). The synthesis is outlined in Scheme 4.

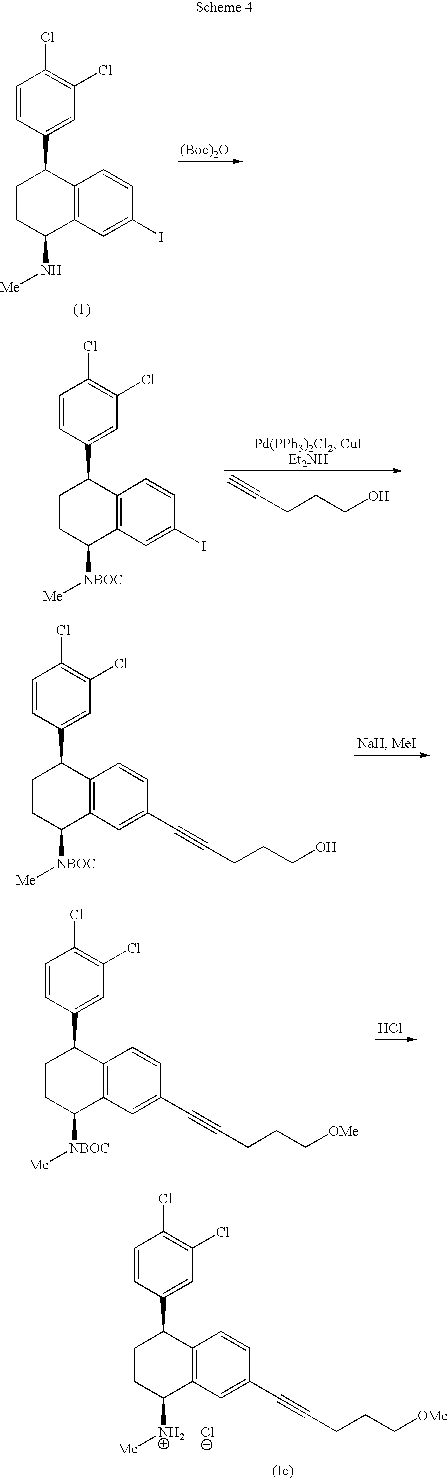

### (1S-cis)-4-(3,4-dichlorophenyl)-7-iodo-1,2,3,4-tetrahydro-N-tert-butoxycarbonyl-N-methyl-1-naphtalenamine

Di-tert-butyl dicarbonate (0.42 g, 1.9 mmol) was added to a solution of a compound represented by formula (1) (0.76 g, 1.8 mmol) and diisopropylethylamine (0.33 mL, 1.8 mmol) in 20 mL dichloromethane under nitrogen. After 22 h the reaction mixture was washed with aqueous citric acid solution (3×20 mL). The aqueous phase was extracted with 20 mL dichloromethane and the combined organic phase was then washed with brine (20 mL) and dried over Na₂SO₄. Dichloromethane was evaporated to yield 0.69 g (74%) of crude brownish oil, which was used without further purification for the next step. In the NMR spectrum two rotamers are observable (minor rotamer data in square parentheses). ¹H NMR (400 MHz, CDCl₃): δ 7.42 (br s, 1H), 7.38 (d, J=8.0 Hz, 1H), 7.22 (d, J=8.4 Hz, 1H), 6.97 (br s, 1H), 6.68 (br s, 1H), 6.58 (d, J=8.0 Hz, 1H), 5.29 (br s, 1H), [5.12 (br s, 1H)], 4.01 (m, 1H), 2.53 (s, 3H), 2.12 (m, 1H),1.88 (m, 1H), 1.60 (m, 2H), 1.42 (s, 9H). ¹³C NMR (100 MHz, CDCl₃): δ 156.4, 146.6, 146.3, 139.1, 137.7, 136.2, 136.0, 132.3, 131.9, 130.4, 130.0, 127.8, 92.8, 80.0, 54.7, 53.7, 42.6, 29.8, 28.3, [21.7], 21.2. MS (FAB): calcd. for C₁₇H₁₇Cl₂IN ((M-BOC)H₂⁺) 432.0; found 431.9.

### (1S-cis)-4-(3,4-dichlorophenyl)-7-(5-hydroxy-1-pentyn-1-yl)-1,2,3,4-tetrahydro-N-tert-butoxycarbonyl-N-methyl-1-naphtalenamine

(1S-cis)-4-(3,4-dichlorophenyl)-7-iodo-1,2,3,4-tetrahydro-N-tert-butoxycarbonyl-N-methyl-1-naphtalenamine (0.69 g, 1.3 mmol), Pd(PPh₃)₂Cl₂ (0.02 g, 0.26 mmol), CuI (0.01 g, 0.055 mmol), 4-pentyn-1-ol (0.13 mL, 1.4 mmol), diethylamine (2.2 mL, 21 mmol) and dry DMF (4 mL) were mixed and stirred under nitrogen overnight. The solvent was removed under reduced pressure and the resulting crude brown oil was dissolved in ethyl acetate (15 mL) and washed with a saturated aqueous solution of potassium carbonate (15 mL). The organic phase was washed with brine (15 mL) and dried over MgSO₄. Ethyl acetate was evaporated under reduced pressure and the resulting crude yellow oil was purified on silica gel (eluent:ethyl acetate:hexanes, 0:100 to 30:70) to yield 0.23 g (36%) of brown oil. In the NMR spectrum two rotamers are observable (minor rotamer data in square parentheses). ¹H NMR (400 MHz, CDCl₃): δ 7.17 (d, J=8.0 Hz, 1H), 7.10 (br m, 1H), 7.05 (d, J=8.0 Hz, 1H), 6.91 (br s, 1H), 6.71 (d, J=8.0 Hz, 1H), 6.66 (br m, 1H), 5.27 (br m, 1H), [5.10 (br m, 1H)], 3.99 (m, 1H), 3.65 (t, J=6.2 Hz, 2H), 2.49 (br s, 3H), 2.39 (t, J=7.0 Hz, 2H), 2.27 (s, 1H), 2.10 (m, 1H), 1.84 (m, 1H), 1.72 (quip, J=6.4 Hz, 2H), 1.58 (m, 2H), 1.38 (s, 9H). ¹³C NMR (100 MHz, CDCl₃): δ 156.3, 146.6, 146.5, 137.8, [137.4], 136.6, 132.2, 130.5, 130.4, 130.3, 130.2, 129.9, 127.8, 122.6, 89.3, 80.5, 79.7, 61.6, 54.9, 53.8, 42.8, 31.2, 29.9, 28.3, [21.9], 21.4, 15.8. MS (FAB): calcd. for C₂₂H₂₄Cl₂NO ((M-BOC)H₂⁴) 388.1; found 388.1.

### (1S-cis)-4-(3,4-dichlorophenyl)-7-(5-methoxy-1-pentyn-1-yl)-1,2,3,4-tetrahydro-N-tert-butoxycarbonyl-N-methyl-1-naphtalenamine

A solution of (1S-cis)-4-(3,4-dichlorophenyl)-7-(5-hydroxy-1-pentyn-1-yl)-1,2,3,4-tetrahydro-N-tert-butoxycarbonyl-N-methyl-1-naphtalenamine (0.12 g, 0.24 mmol) in 0.36 mL of dry THF, was added to sodium hydride (0.035 g, 0.72 mmol; 55%-65% in mineral oil) at 0° C. in a pressure tube under nitrogen. The mixture was stirred for 30 min, followed by the addition of methyl iodide (0.05 mL, 0.72 mmol) in 0.24 mL of dry THF. The tube was sealed and, after 5 min, the reaction mixture was allowed to worm to room temperature and then brought to 60° C. After 3 days, excess sodium hydride was destroyed by the dropwise addition of a saturated aqueous solution of ammonium chloride to the cooled (0° C.) reaction mixture and the resulting solution was extracted three times with ethyl acetate (5 mL). The combined organic extracts were washed with a saturated aqueous solution of ammonium chloride and then with brine (15 mL each), and dried over MgSO₄. The ethyl acetate was evaporated under reduced pressure and the crude oil purified on silica gel (eluent:ethyl acetate:hexanes, 0:100 to 5:95) to yield 40 mg (33%) of brown oil. In the NMR spectrum two rotamers are observable (minor rotamer data in square parentheses). NMR (400 MHz, CDCl₃): δ 7.32 (d, J=8.4 Hz, 1H), 7.24 (br s, 1H), 7.21 (d, J=8.0 Hz, 1H), 7.05 (br s, 1H), 6.87 (d, J=8.0 Hz, 1H), 6.79 (br m, 1H), 5.42 (br m, 1H), [5.24 (br m, 1H)], 4.15 (m, 1H), 3.53 (t, J=6.2 Hz, 2H), 3.37 (s, 3H), 2.62 (br s, 3H), 2.51 (t, J=7.0 Hz, 2H), 2.24 (m, 1H), 1.98 (m, 1H), 1.87 (quin, J=6.6 Hz, 2H), 1.72 (n, 2H), 1.52 (s, 9H). ¹³C NMR (100 MHz, CDCl₃): δ 156.1, 146.6, 146.5, 137.4, [137.1], 136.4, 131.9, 130.2, 130.2, 130.0, 129.9, 129.7, 127.7, 122.7, 89.2, 80.3, 79.4, 70.8, 58.1, 54.2, 53.6, 42.6, 29.7, 28.4, 28.1, [21.7], 21.2, 15.7.

### (1S-cis)-4-(3,4-dichlorophenyl)-7-(5-methoxy-1-pentyn-1-yl)-1,2,3,4-tetrahydro-N-methyl-1-naphthalenamine hydrochloride (Ic)

(1S-cis)-4-(3,4-dichlorophenyl)-7-(5-methoxy-1-pentyn-1-yl)-1,2,3,4-tetrahydro-N-tert-butoxycarbonyl-N-methyl-1-naphtalenamine (0.04g, 0.08 mmol) was dissolved in 2 mL of 4M HCl in dioxan. After 3 h the solvent was removed in vacuo to yield 18 mg (52%) of the compound represented by formula (Ic) as an off-white solid. ¹H NMR (400 MHz, CD₃OD): δ 7.58 (s, 1H), 7.46 (d, J=8.0 Hz, 1H), 7.41 (s, 1H), 7.28 (d, J=8.0 Hz, 1H), 7.18 (br d, J=7.7 Hz, 1H), 6.83 (d, J=8.0 Hz, 1H), 4.45 (br s, 1H), 4.12 (br m, 1H), 3.50 (t, J=6.0 Hz, 2H), 3.32 (s, 3H), 2.81 (s, 3H), 2.46 (t, J=7.0 Hz, 2H), 2.26 (m, 1H), 2.14 (m, 2H), 1.98 (m, 11-1), 1.80(quin, J=6.4 Hz, 2H). MS (FAB): calcd. for C₂₃H₂₆Cl₂NO (MH+) 402.1; found 402.1.

### Example 4

This example illustrates a proposed synthesis of an exemplary compound of the present invention. The synthesis is outlined in Scheme 5.

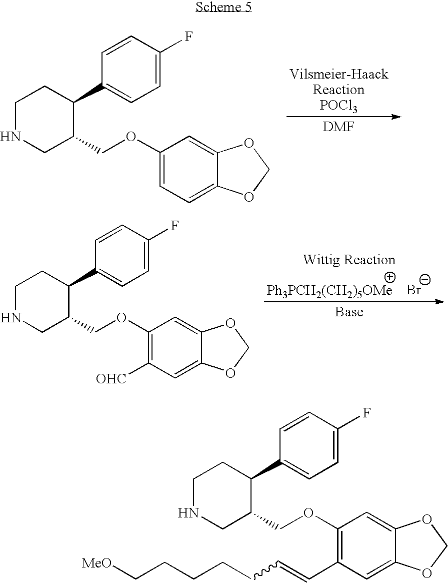

### Example 5

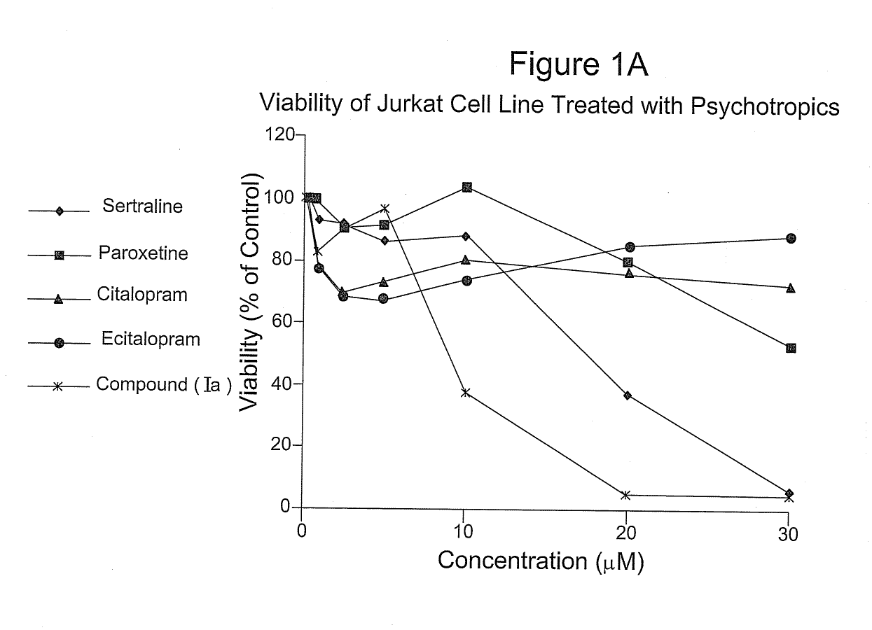

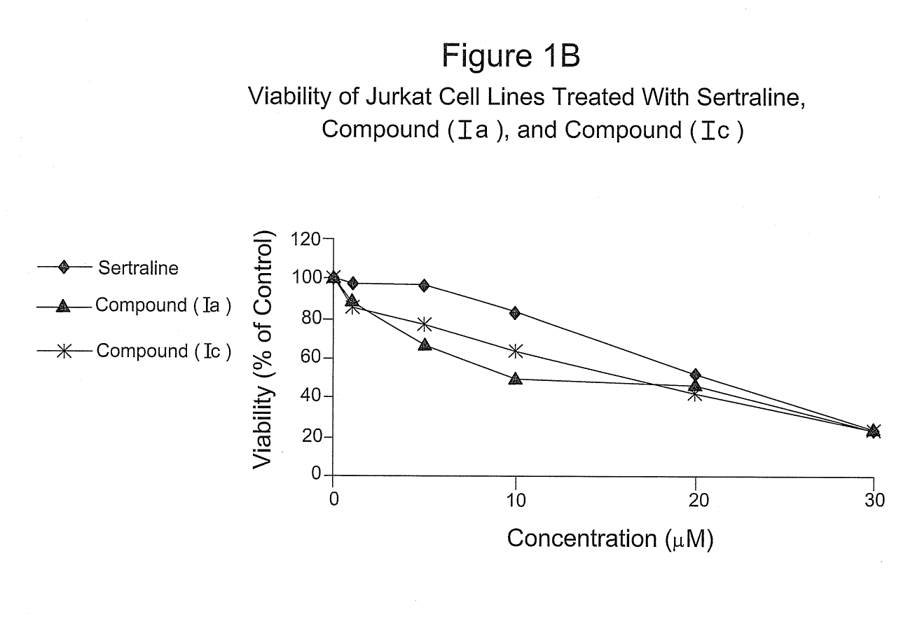

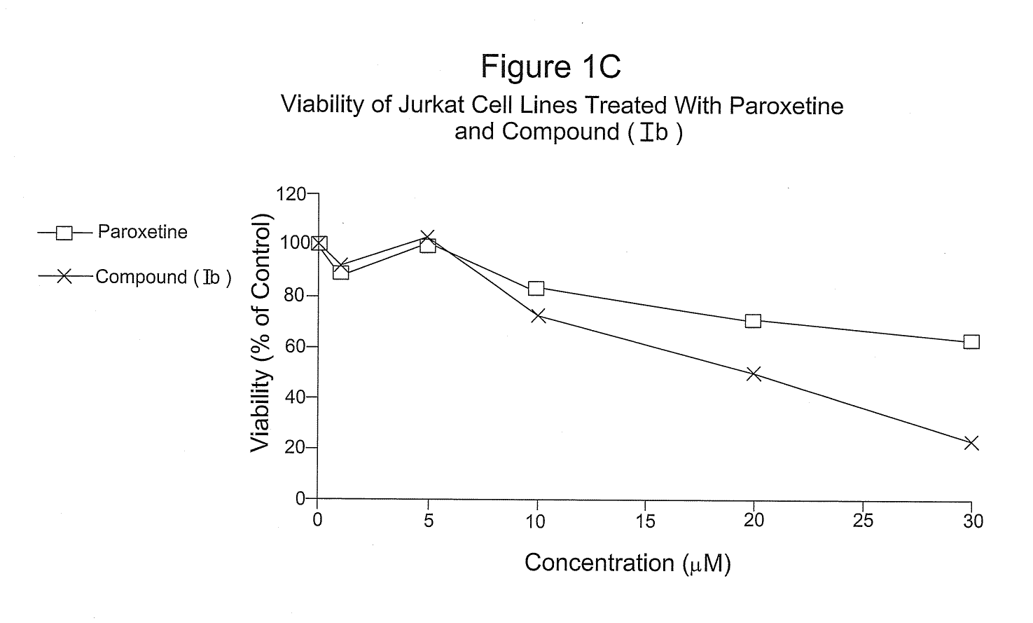

This example illustrates cell viability data derived from the incubation of a human. Jurkat lymphoma cell-line with different concentrations of the compounds represented by formulae (Ia), (Ib), and (Ic) (hereinafter “compound (Ia),” “compound (Ib),” and “compound (Ic),” respectively). The results are depicted in FIGS. 1A, 1B, and 1C. Cells 10000/well were incubated with sertraline, paroxetine, citalopram and its isomer ecitalopram, and compared to the effect of compound (Ia), compound (Ib), and compound (Ic) (2.5-30 μM) on viability of human lymphoma cells 48 hr after exposure using alamar blue staining method. See Nociari et al., J. Immunol. Methods, 15 (213), 157-167 (1998). Each point represents the mean+/−SE of 4 determinations.

The data in FIG. 1A reveals that the antidepressants have a differential effect on the viability of human Jurkat lymphoma cell-line. The two isomers of citalopram, citalopram and ecitalopram, caused a slight decrease in cell-viability at low concentrations (2.5 and 5.0 μM), while paroxetine and sertraline exhibited a dose dependant inhibition in cell viability. Paroxetine, sertraline, and compound (la) exhibited anti-proliferative activity with IC₅₀ values of 37.7 μM,16.8 μM, and 12.5 μM, respectively.

The data in FIG. 1B reveals that sertraline, compound (Ia), and compound (Ic) caused a dose dependent decrease in cell viability with IC₅₀ levels of 20.1, 16.2 and 17.0 μM, respectively. Thus the data reveals that compound (Ia) and compound (Ic) exhibited higher potency than sertraline at the lower concentrations (1-10 μM).

The data in FIG. 1C reveals that paroxetine induced a slight antiproliferative effect on human Jurkat Lymphoma cells, while compound (Ib) caused a greater amount inhibition of cell viability with IC₅₀ levels of 37.6 and 19.5 μM for paroxetine and compound (Ib), respectively. Thus the data reveals that compound (Ib) exhibited higher potency than paroxetine at concentrations higher than 5 μM.

### Example 6

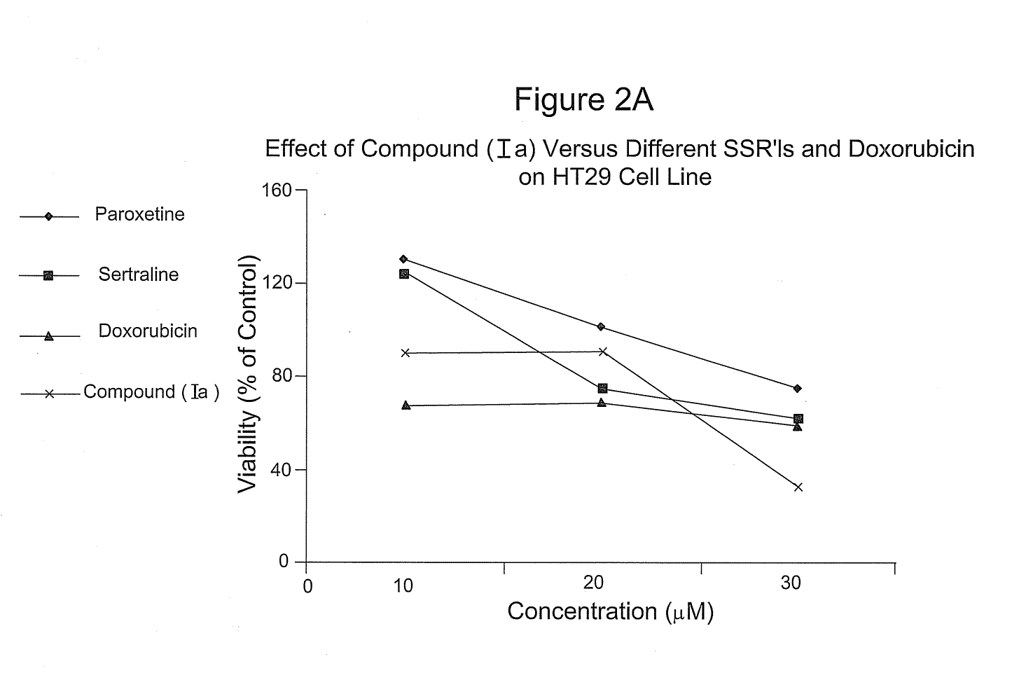

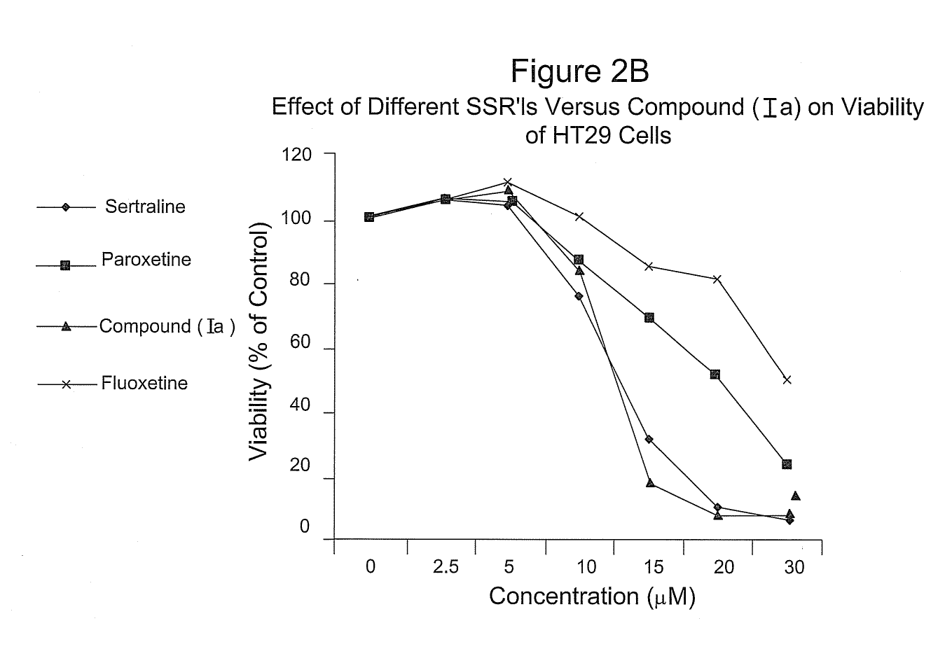

This example illustrates cell viability data obtained by administering different concentrations of compound (Ia) to human colon carcinoma cells (HT29). The results are depicted in FIG. 2A. This graph comparatively depicts the effect of paroxetine, sertraline (10-30 μM), the chemotherapeutic agent doxorubicin, and compound (Ia) on the viability of human colon carcinoma cells (HT29) using neutral red staining Each point represents the mean+/−SE of 4 determinations. See Borenfreund et al., J. Tissue Culture Methods 9, 7-9 (1984). The results reveal the existence of a dose dependent decrease in cell viability, with sertraline and compound (Ia) exhibiting higher potency than paroxetine. At concentrations of 10 and 20 μM the effect of doxorubicin, in terms of cell viability, was more pronounced than that produced by the antidepressants and compound (Ia). However, at 30 μM, compound (Ia) yielded a cell viability of 30.9% of controls, as compared to 57.7% for doxorubicin, 60.3% for sertraline, and 74% for paroxetine.

### Example 7

This example illustrates cell viability data obtained by administering different concentrations of compound (Ia), sertraline, paroxetine, and fluoxetine to human colon carcinoma cells (HT29). FIG. 2B depicts the effect of sertraline, paroxetine, fluoxetine and compound (Ia) at equimolar doses on viability of human colon cancer cells HT29. The data indicates that sertraline and compound (Ia) were significantly more potent than paroxetine and fluoxetine. In fact, sertraline and compound (Ia) provided an IC₅₀ value of 12.7 μM.

### Example 8

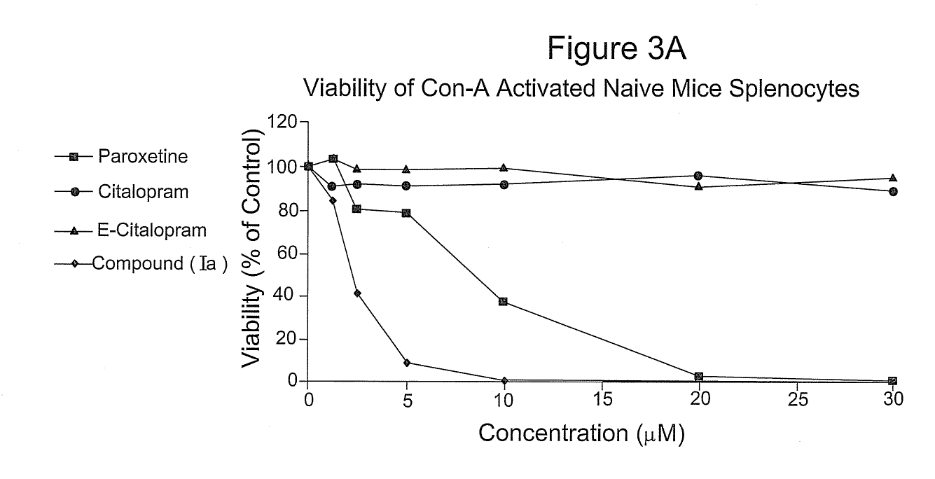

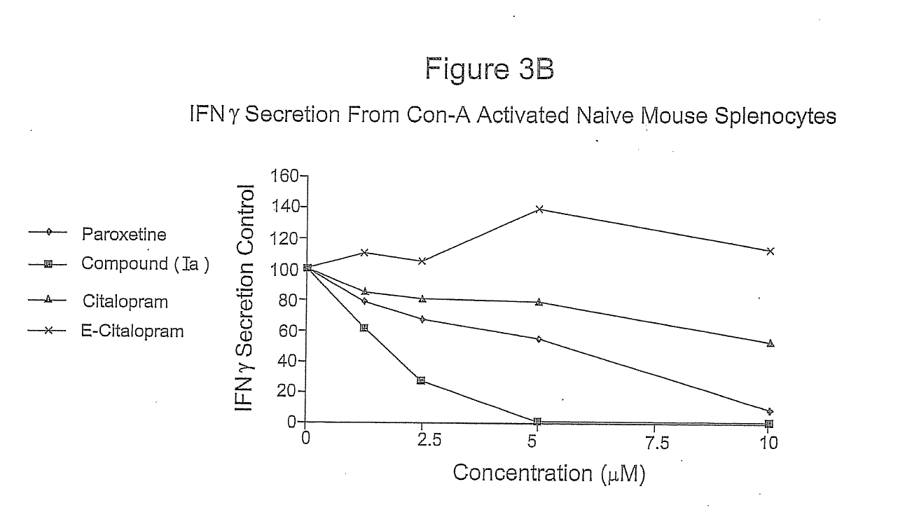

This example illustrates cell viability data obtained by separately administering different concentrations of compound (Ia), paroxetine, citalopram, and ecitalopram to Con-A activated naive mouse splenocytes. FIG. 3A depicts the comparative effect of compound (Ia), paroxetine, citalopram and ecitalopram on Con-A-induced mouse splenocyte proliferation using alamar blue staining after 48 hr. Each point represents the mean+/−SE of 5 determinations. The results show that the antidepressants have a differential on healthy splenocytes proliferation. The two isomers of citalopram had no effect on cell-viability up to 30 μM, while paroxetine induced a dose dependent inhibition in cell viability (IC₅₀ 11.9 μM) and compound (Ia) induced a higher inhibitory activity of IC₅₀ 3.9 μM. These results suggest that compound (Ia) is a more potent immunomodulating agent than paroxetine, citalopram, and ecitalopram.

### Example 9

This example illustrates IFN y secretion data obtained by separately administering different concentrations of compound (Ia), paroxetine, citalopram, and ecitalopram to Con-A activated naive mouse splenocytes. The results are depicted in FIG. 3B. The data shows that compound (Ia) exhibited greater IFN γ secretion inhibitory activity than paroxetine, citalopram, and ecitalopram.

### Example 10

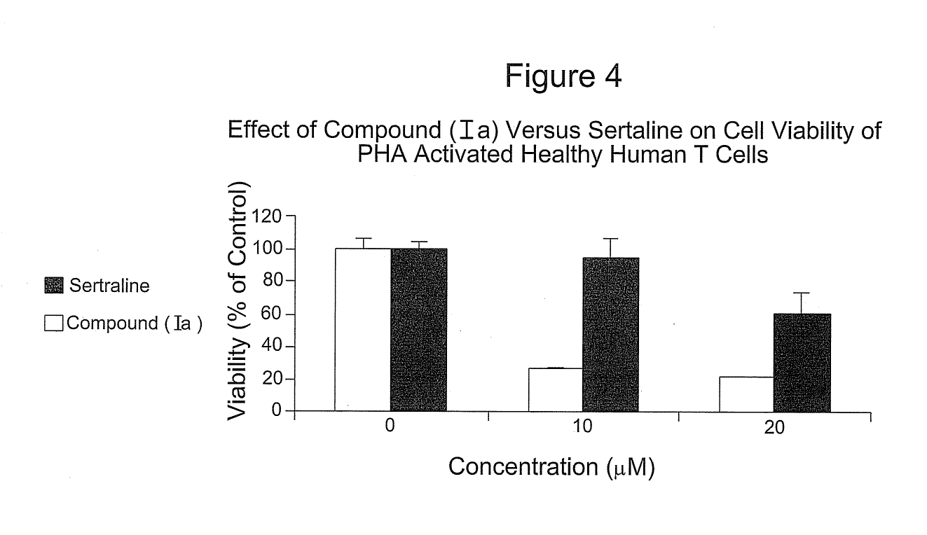

This example illustrates cell viability data obtained by separately administering different concentrations of compound (Ia) and sertraline to PHA activated healthy human T cells. The results are depicted in FIG. 4, which compares the effect of compound (Ia) and sertraline on PHA induced proliferation of healthy human lymphocytes using alamar blur staining after 48 hr. Each point represents the mean+/−SE of 5 determinations. The data indicates that in human PHA activated healthy lymphocytes, sertraline and compound (Ia) inhibit cell proliferation. However, the potency of compound (Ia), IC₅₀ 9.8, is about 3 times greater than that of sertraline, IC₅₀ 27.6.

### Example 11

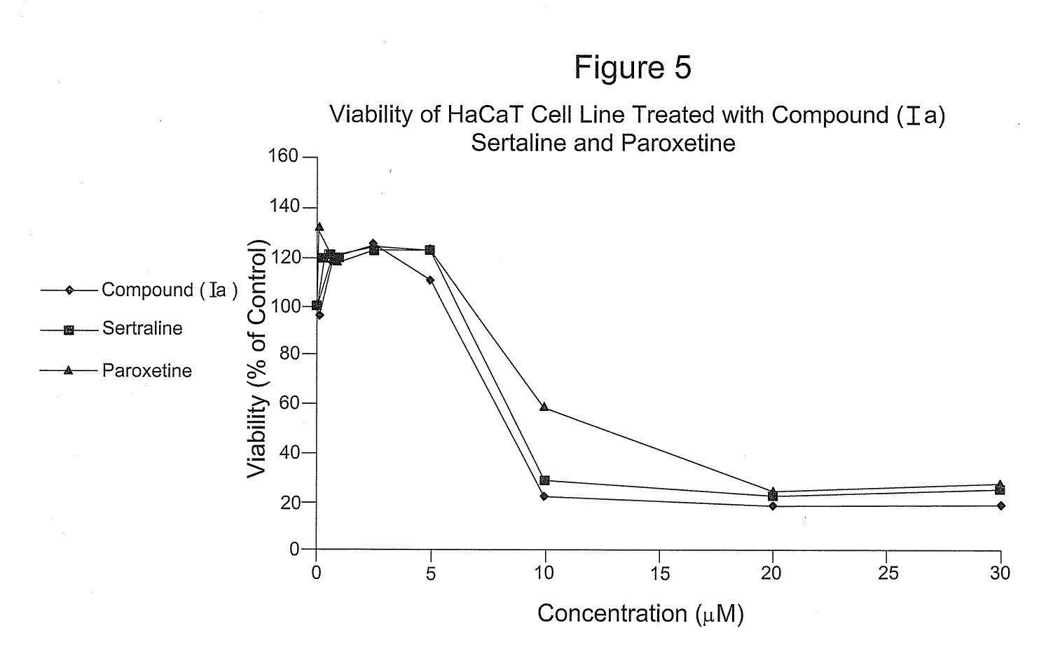

This example illustrates cell viability data obtained by separately administering different concentrations of compound (Ia), sertraline, and paraoxetine to HaCat cell line. The results are depicted in FIG. 5, which compares the effect of compound (Ia), paroxetine, and sertraline on the viability of human keratinocytes (HaCat cells) using neutral red staining after 24 hr. Each point represents the mean+/−SE of 3 determinations. Based on these results, it is apparent that paroxetine, sertraline and compound (Ia) all induce a dose dependent decrease in cell viability, at concentrations higher than 5 μM. However, compound (Ia) had a higher activity, with an IC₅₀ value of 18.5 μM, compared to sertraline and paroxetine, which had IC₅₀ values of 13.7 and 16.0, respectively.

### Example 12

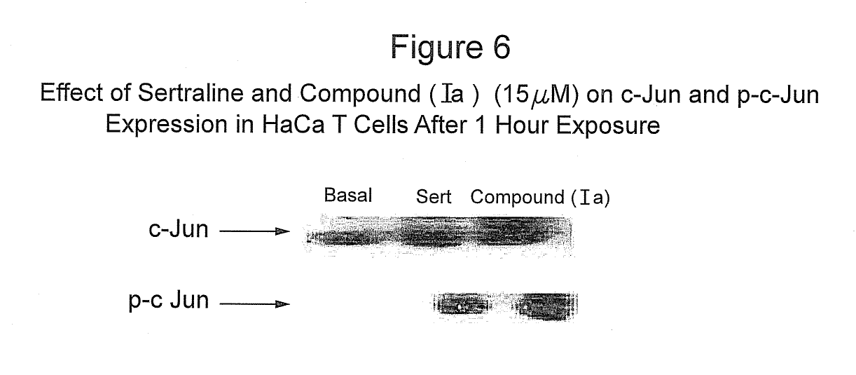

This example illustrates western blot data corresponding to c-jun and p-c-jun expression in HaCat cells treated with different concentrations of compound (Ia) and sertraline. The results are shown in FIG. 6. The HaCat cells were exposed to compound (II) (15 μM) and sertraline (15 μM) for 1 hr. For each sample 2×107 cells were used. The cells were lysed, and equal amounts of protein was fractionated on polyacrylamide gel and then transferred to PVDF membrane. The amount of the protein was detected by immuno-blotting with monoclonal anti-c-Jun or p-c-Jun antibodies (Cayman, Ann Arbor, Mich., USA). A second antibody conjugated to horseradish peroxidase was used and visualized with chemiluminescent Kit (Pierce, Rockford, Ill., USA) on film. The results indicate that sertraline and compound (Ia), both at 15 μM, affect human keratinocytes by rapid activation of the MAPK pathway and specifically by activation of the c-Jun-N-terminal kinase (JNK) pathway. Activation of the WIC pathway produces p-c-Jun as a major end, while initiating apoptosis in certain cell-lines.

### Example 13

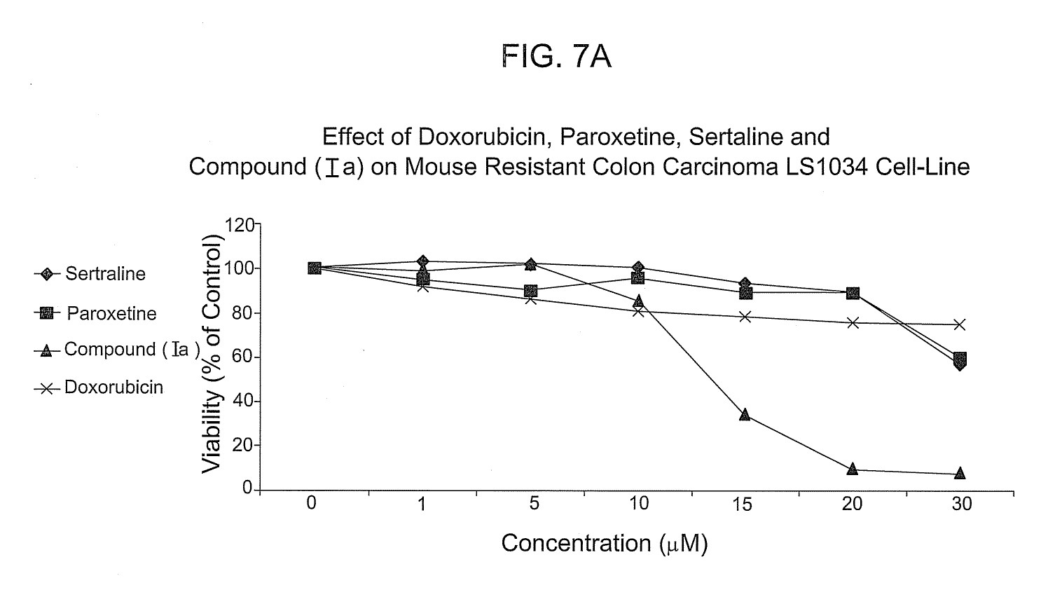

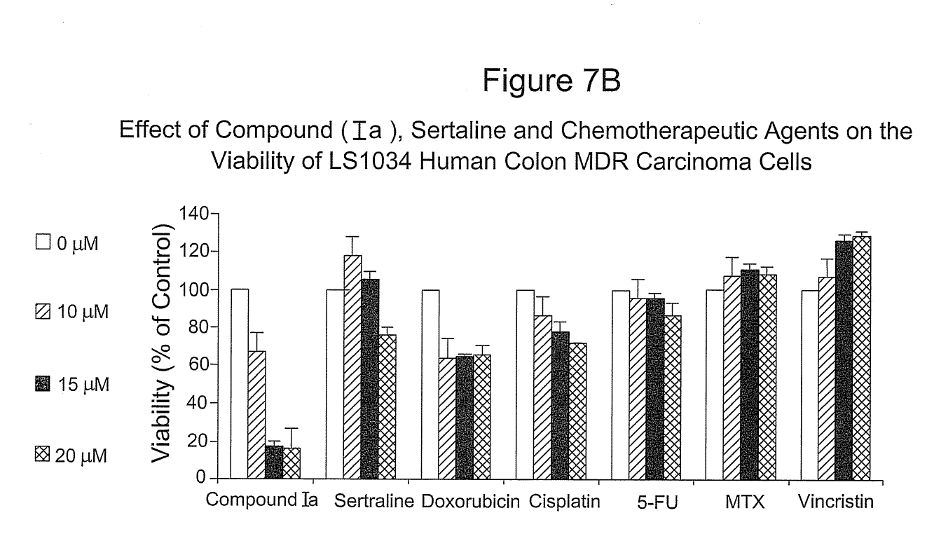

This example illustrates cell viability data obtained by separately administering different concentrations of compound (Ia), sertraline, paroxetine, and doxorubicin to a mouse resistant colon carcinoma LS1034 cell-line. The results are depicted in FIG. 7A. These results show that compound (Ia) exhibited significant inhibition of cell viability, with an IC₅₀ 11.3 μM, compared to doxorubicin, sertraline and paroxetine.

### Example 14

This example illustrates cell viability obtained by separately administering different concentrations of compound (Ia), sertraline, doxorubicin, cisplatin, fluorouracil (5-FU), methotrexate (MTX), and vincristin to LS1034 human colon MDR carcinoma cells. The results are depicted in FIG. 7B. These results suggest that compound (Ia) inhibits the cell efflux transporter P-glycoprotein (Pgp) and, thus has the ability to suppress resistant cancer cells.

### Example 15

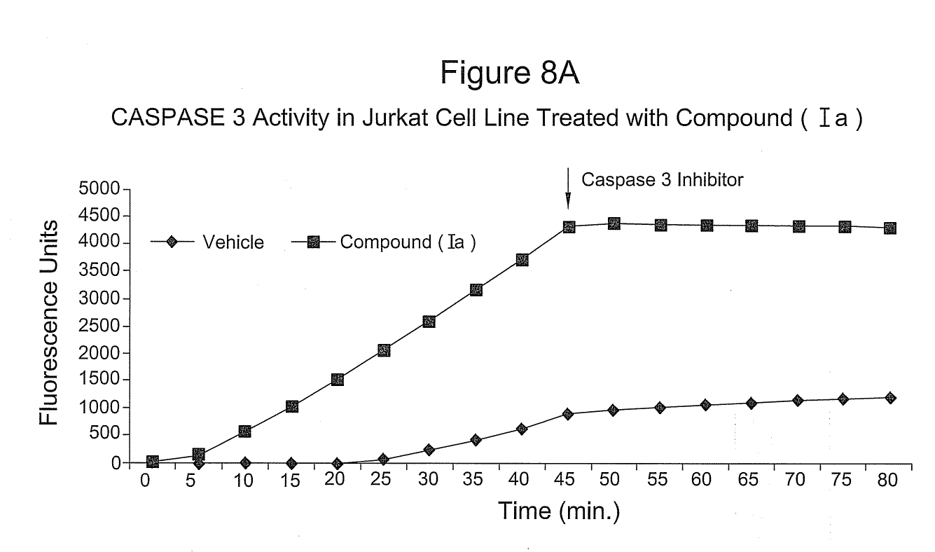

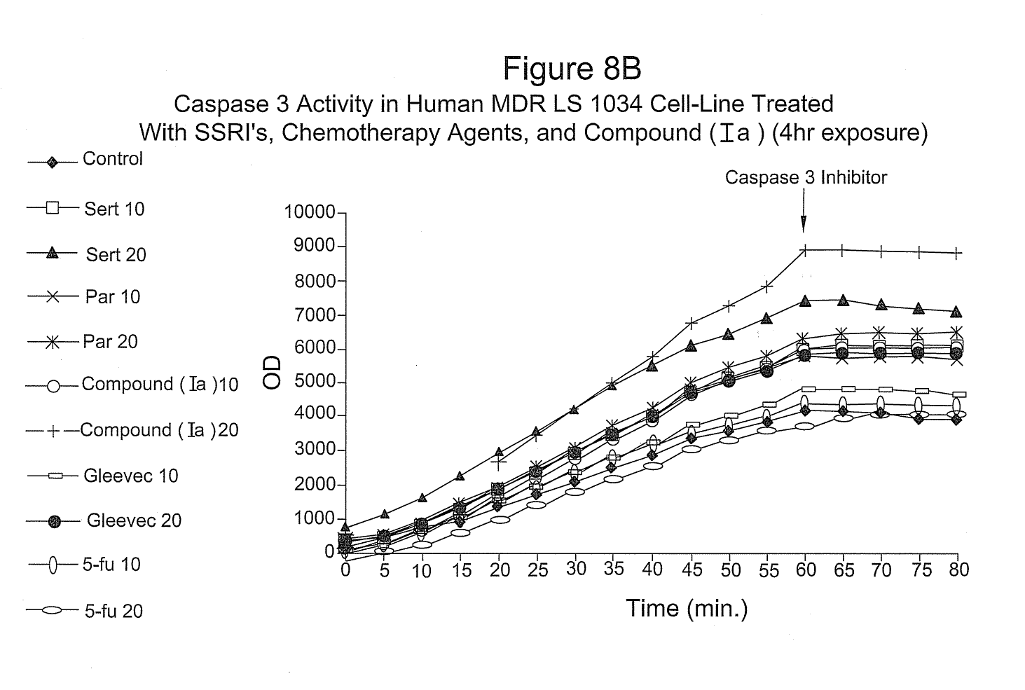

This example illustrates Caspase 3 activity data obtained by administering compound (Ia) to a human Jurkat lymphoma cell-line. The results are depicted FIG. 8A. Each point is the mean of 2 determinations.

Caspase 3 activity was measured by an enzymatic fluorimetric method using a fluorigenic substrate (Ac-DEVD-AMC), which produces blue fluorescence detected at 360 nm wavelength. See Garcia-Calvo et al., J Biol Chem, 273(49), 32608-13 (1998). When AMC is cleaved from the substrate by caspase-3, and caspase-3-like enzymes, it produces a yellow-green fluorescence, which can be monitored by a fluorimeter at 460 nm. The amount of yellow green fluorescence is proportional to the activity of caspase-3 in the cell extract sample.

The human Jurkat lymphoma cell-line, as used in FIG. 8A, and the human MDR colon cancer LS1034 lysates, as used in FIG. 8B, were prepared by Triton X-100 extraction 4 hr after exposure to the applicable compound (10 or 20 μM) or the vehicle. Whole cell lysate was added to a buffer containing 100 μM peptide substrate, 100 mM HEPES, 10% glycerol, 1 mM EDTA and 10 mM dithiothreitol. Measurements were taken every 5 min for 80 min. These experiments were also monitored in the presence of a specific caspase-3 inhibitor, DEVD-AMC-CHO, which was added after 45 or 60 min to the reaction mixture, to ascertain the specificity of the enzyme.

The results show that compound (Ia) provided an increase in the activity of the caspase-3 as compared to vehicle treated cells. Since caspase-3 is an early marker of apoptosis in cells, this data suggests that the apoptotic mechanism in lymphoma Jurkat cells has been activated.

### Example 16

This example illustrates caspase-3 activity data derived from the treatment of LS1034 human colon MDR carcinoma cells with compound (Ia) and a variety of selective serotonin reuptake inhibitors and chemotherapy (e.g., anti-cancer) drugs. The results are depicted in FIG. 8B. In this case, the results show a dose dependent increase in the activity of caspase-3 activity for compound (Ia), sertraline, paroxetine, and gleevec. These results suggest that compound (Ia) effectively activates caspase-3 also in MDR cancer cells.

### Example 17

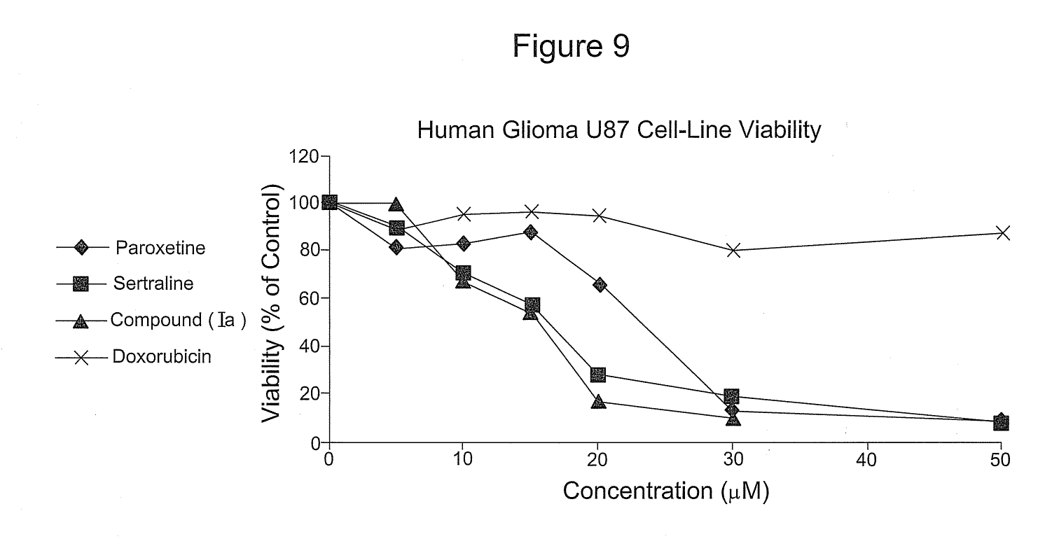

This example illustrates cell viability data derived from the treatment of a human glioma U87 cell-line with different concentrations of compound (Ia), paroxetine, sertraline, and doxorubicin. The results are depicted in FIG. 9. Each point represents the mean+/−SE of 3 determinations. The data shows that paroxetine, sertraline and compound (Ia) inhibit this glioma cell-line in a dose dependant fashion. Moreover, compound (Ia) and sertraline appear to have a higher efficacy than paroxetine.

### Example 18

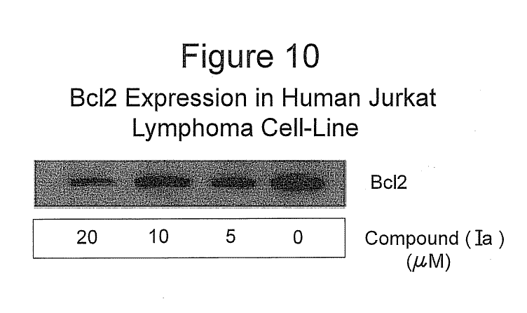

This example illustrates western blot data corresponding to BCL2 expression in a human Jurkat lymphoma cell-line treated with different concentrations of compound (Ia). The results are depicted in FIG. 10. Treatment of the lymphoma cells with compound (Ia) induced a rapid dose dependent decrease in the levels of the protooncogene Bcl2. Since Bcl2 is an important stimulatory factor in cell proliferation and in a variety of cancer pathologies, a decrease in its expression suggests that compound (Ia) possesses anti-cancer activity.

### Example 19

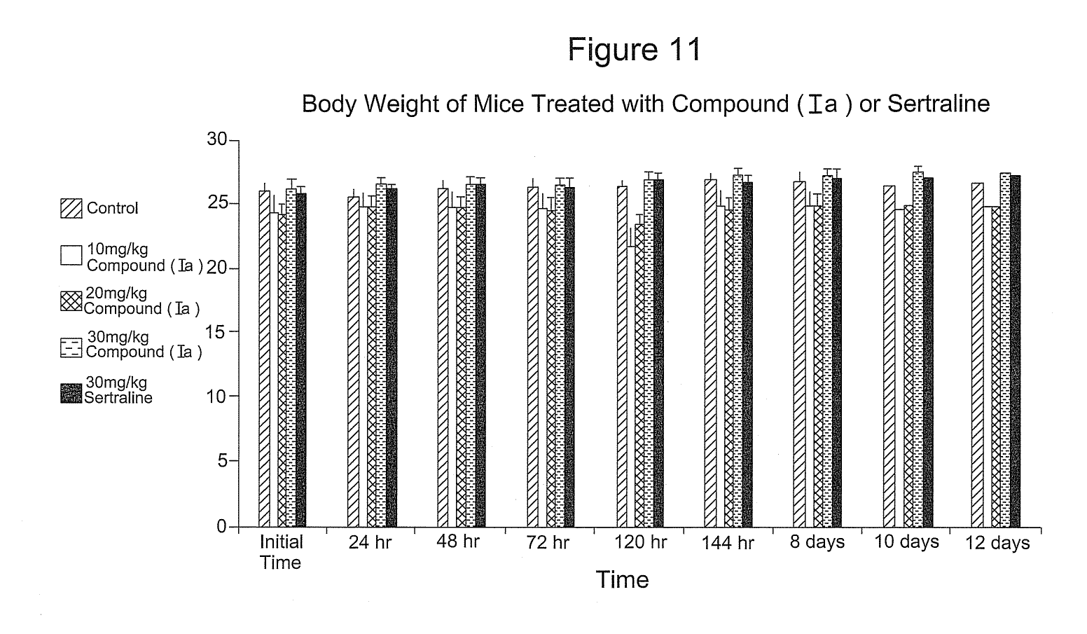

This example illustrates acute toxicity data derived from the treatment of male Balb/C mice with different concentrations of compound (Ia). The results are depicted in FIG. 11. Compound (Ia) was administered ip to groups of mice at doses of 10, 20 and 30 mg/kg. Sertraline was also administered to one group at 30 mg/kg. Each group of mice included five animals. Based on these results, it is evident that compound (Ia) inhibits abnormal or undesirable cell proliferation.

The animals were observed for behavioral changes such as stimulation, aggression, sedation, social distribution for 4 h and then after 24 h. Body weight was determined up to 12 days after drug administration. The results indicate that acute ip administration of compound (Ia) of up to 30 mg/kg was well tolerated. In fact, none of the animals showed any observed change in behavior. In addition, none of the animals exhibited changes in food intake or body weight over the 12 days in which this experiment was performed.

### Example 20

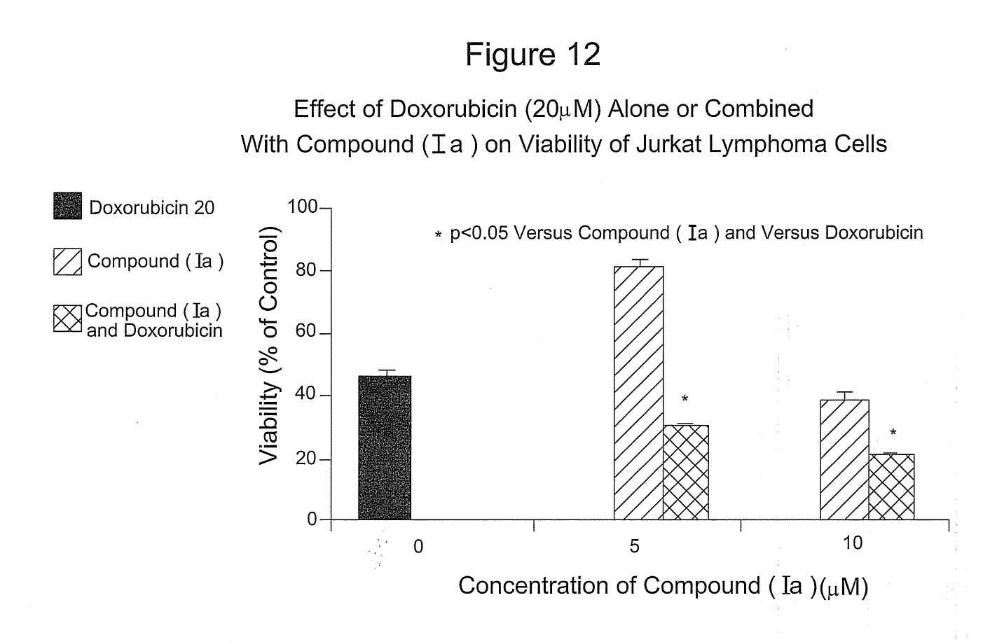

This example illustrates cell viability data derived from the treatment of Jurkat Lymphoma cells with Doxorubicin (20 μM), compound (Ia) (5 and 10 μM), and the combination of both Doxorubicin and compound (Ia). The results are depicted in FIG. 12. Doxorubicin alone inhibited cell viability by 54%, compound (la) by 18% and 59.5% respectively for 5 and 10 μM. Co-administration of a solution of Doxorubicin (20 μM) and a solution of compound (Ia) (5 μM) to Jurkat Lymphoma cells resulted in an improved inhibition value of 69%. Co-administration of a solution of a solution of Doxorubicin (20 μM) and a solution of compound (Ia) (10 μM) to Jurkat Lymphoma cells resulted in an improved inhibition value of 77%. The results indicate that the using doxorubicin in combination with compound (Ia) results in an additive effect with regard to inhibition of cancer cells.

### Example 21

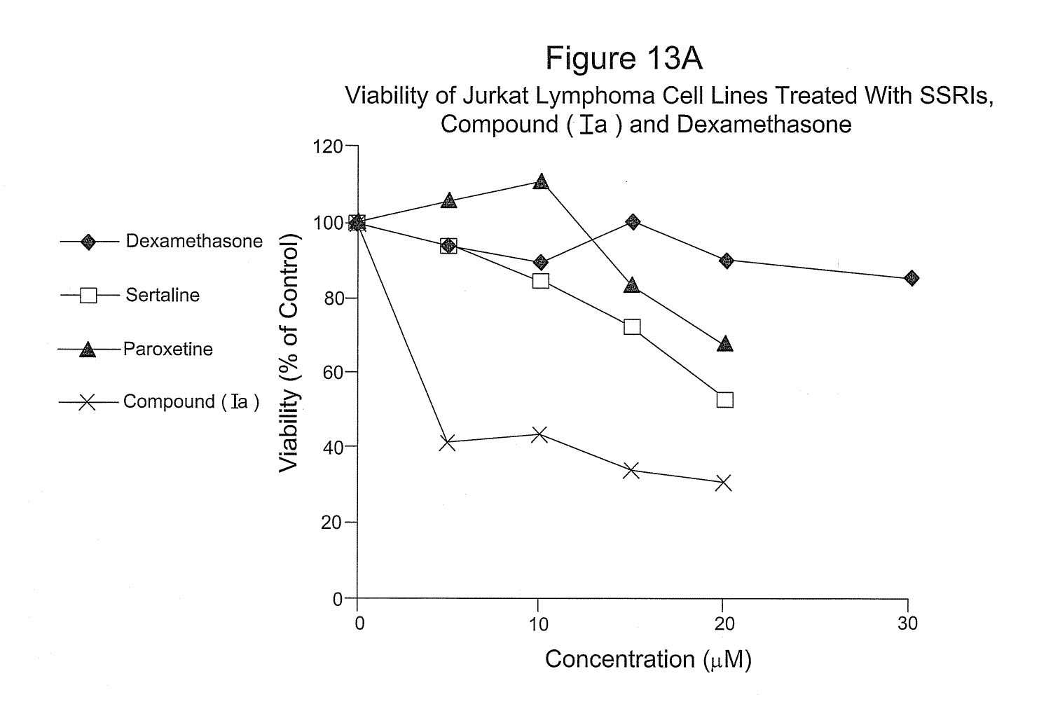

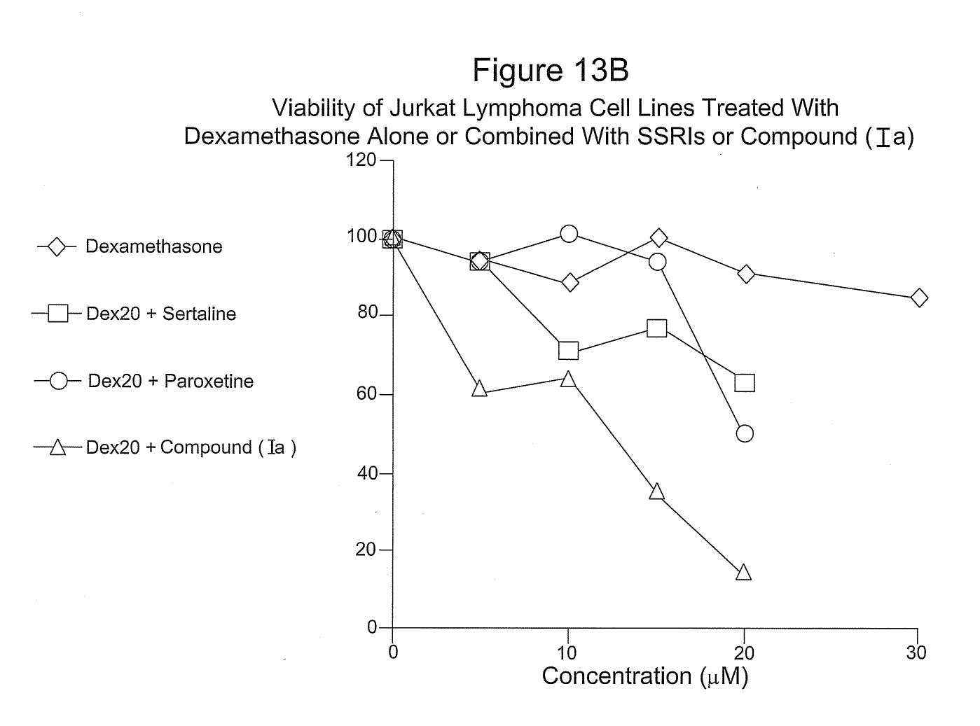

This example illustrates cell viability data obtained by separately administering different concentrations of compound (Ia), sertraline, paroxetine, and the corticosteroid dexamethasone to human Jurkat lymphoma cell-lines. The results are depicted in FIG. 13A. The results show that the lymphoma cells demonstrated a resistance to dexamethasone in terms of cell viability. Compound (Ia) provided inhibition of cell viability that was higher than that produced by either sertraline or paroxetine. Thus the results indicate that the compounds of the present invention may act as anti-proliferative agents in hematological and other cancer cells resistant to corticosteroids.

The results depicted in FIG. 13B indicate that dexamethasone in combination with sertraline, paroxetine, and compound (Ia) provide inhibition of cell viability in Jurkat lymphoma cells. The results show that the combination of dexamethasone with compound (Ia) inhibited cell viability more successfully than either dexamethasone or compound (Ia) alone. For example, dexamethasone (20 μM) inhibited cell viability by 8%, compound (Ia) (20 μM) inhibited cell viability by 69%, and the combination inhibited cell viability by 86%. The results indicate the presence of a possible additive effect resulting from the combination of corticosteroids and the compounds of the present invention for inhibition of proliferative and/or inflammatory disorders.

### Example 22

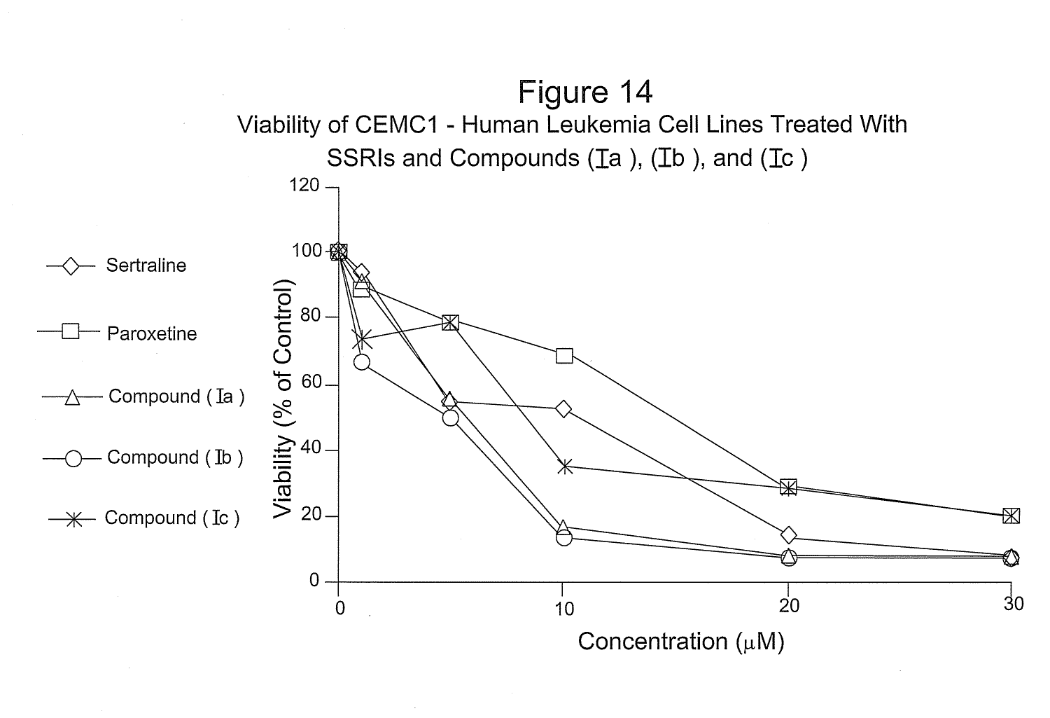

This example illustrates cell viability and proliferation data obtained by separately administering setraline, paroxetine, and compounds (Ia), (Ib), and (Ic) to CEM/C1 cell-lines. This cell-line is a camptothecin (CPT) resistant derivative of the human T-cell leukemia purchased from ATCC (USA). Cells, 10,0000/well, were exposed to setraline, paroxetine, and compounds (Ia), (Ib), and (Ic) at 1-30 μM and viability was measured using the alamar blue dyeing method. The results are depicted in FIG. 14. Each point represents the mean+/−SEM of 4 determinations. The results show that setraline, paroxetine, and compounds (Ia), (Ib), and (Ic) all induced a dose dependent decrease in cell viability, with compound (Ib) and compound (Ia) being more potent than paroxetine and setraline, respectively (i.e., IC₅₀ values of 7.6 μM and 8.9 μM compared to 12.3 μM and 16.3 μM, respectively). Compound (Ic) had a lower activity compared to compounds (Ia) and (Ib) (IC₅₀ of 13.5 μM).

### Example 23

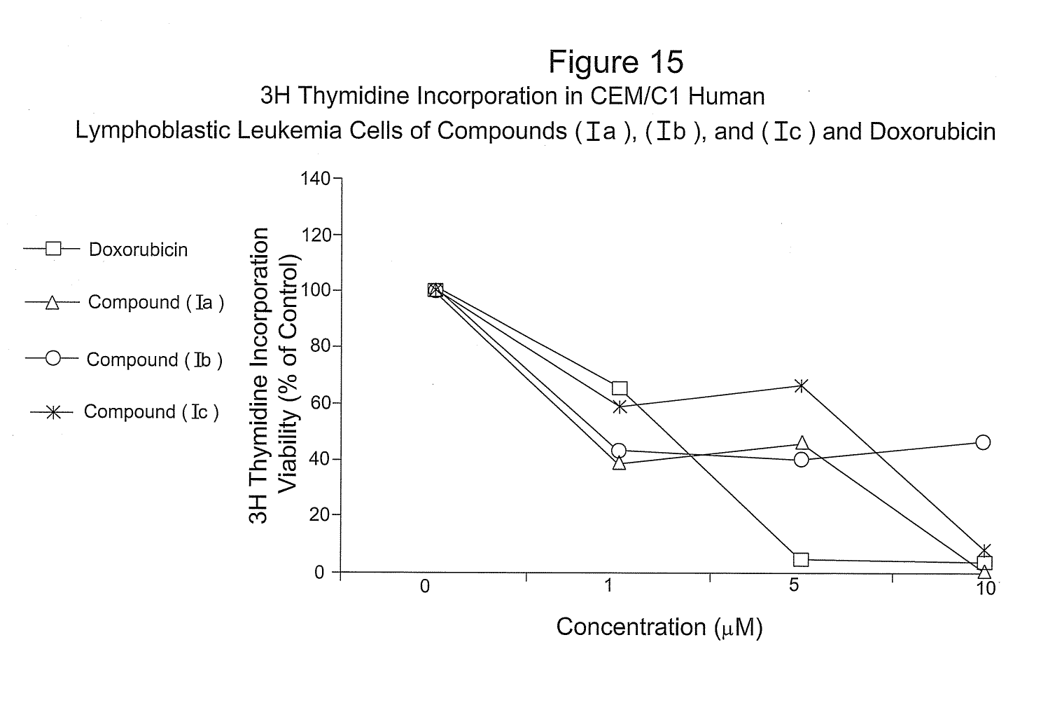

This example illustrates cell proliferation data obtained by separately administering doxorubicin (1-10 μM) and compounds (Ia), (Ib), and (Ic) to CEM/C1 human leukemia cells. This data was collected using the 3H thymidine incorporation method. Thymidine incorporation was measured in cells using 1 μCi/ml H-thymidine for 24 hours. Cells, 10,000/well, were harvested and the incorporated radioactivity was determined by liquid scintillation in a beta counter. The results are depicted in FIG. 15. The results show that compounds (Ia), (Ib), and (Ic) all induced a dose dependent decrease in cell proliferation, which was already evident at 1 μM. Compounds (Ia) and (Ib) showed a higher effect compared to doxorubicin (at 1 μM) (i.e., 38.8 and 43.6% of controls respectively versus 65.7% of control for doxorubicin). At a concentration of 10 μM, doxorubicin, compound (Ia), and compound (Ic) provided cell proliferation inhibitions of about 95%. The results indicate that compounds (Ia), (Ib), and (Ic) are highly effective in the inhibition of cell proliferation, probably via induction of apoptosis. Compounds (Ia) and (Ib) showed significantly higher effects compared to sertraline and paroxetine, and the activity (IC₅₀) of compounds (Ia) and (Ib) is at the same range of known chemotherapeutic agents (e.g doxorubicin).

### Example 24

This example illustrates cell proliferation data in the form of IC₅₀, IC₇₀ and IC₉₀ values obtained by exposure of human cancer cell-lines to compound (Ia). The results, as provided in Table 1, show that compound (Ia) inhibits cell proliferation in 70% of the cell-lines at the concentration of 7.5 In addition, compound (Ia) had a mean IC₅₀ value of 4.9 μM and a mean IC₇₀ value of 6.9 μM. Setraline showed a lower activity with a mean IC₅₀ of 9.1 μM and a mean IC₇₀ of 13 μM, which are about two-fold that of compound (Ia). The results indicate that compound (Ia) is highly effective in the inhibition of cell proliferation.

TABLE 1
In vitro antitumor activity of compound (Ia) in human tumor cell lines
(T/C values).
IN-VITRO ANTITUMOR ACTIVITY OF COMPOUND (Ia) IN
HUMAN TUMOR CELL LINES (Monolayer Assay, PI)

| TUMOR/ | | | | | | | |
|---|---|---|---|---|---|---|---|
| PASSAGE | | CTRL | Test/Control (%) | | | | |
| FLUOR. | EXP. | NO. | at Drug Concentration | | | | |
| NO. | (μg/ml) | UNITS | .75 | 2.3 | 7.5 | 23. | 75. |
| BXF | | | | | | | |
| 1218L | *(3) | 2847 | 101− | 97− | 92− | 4+++ | 5+++ |
| T24 | *(3) | 1893 | 88− | 93− | 53− | 2+++ | 2+++ |
| CNXF | | | | | | | |
| 498NL | *(3) | 3710 | 97− | 101− | 5+++ | 3+++ | 1+++ |
| SF268 | *(3) | 1559 | 91− | 70− | 5+++ | 4+++ | 3+++ |
| CXF | | | | | | | |
| HCT116 | *(3) | 3738 | 99− | 93− | 4+++ | 2+++ | 1+++ |
| HT29 | *(3) | 3943 | 99− | 100− | 3+++ | 1+++ | 1+++ |
| GXF | | | | | | | |
| 251L | *(3) | 1703 | 90− | 80− | 54− | 7+++ | 3+++ |
| HNXF | | | | | | | |
| 536L | *(3) | 1350 | 112− | 102− | 97− | 4+++ | 3+++ |
| LXF | | | | | | | |
| 1121L | *(3) | 3255 | 103− | 104− | 72− | 2+++ | 1+++ |
| 289L | *(3) | 1788 | 94− | 101− | 42+ | 4+++ | 3+++ |
| 526L | *(3) | 1804 | 92− | 81− | 6+++ | 5+++ | 4+++ |
| 529L | *(3) | 1969 | 93− | 97− | 17++ | 3+++ | 5+++ |
| 629L | *(3) | 3233 | 99− | 94− | 4+++ | 1+++ | 2+++ |
| H460 | *(3) | 3413 | 101− | 95− | 2+++ | 1+++ | 1+++ |
| MAXF | | | | | | | |
| 401NL | *(3) | 1960 | 103− | 70− | 9+++ | 3+++ | 3+++ |
| MCF7 | *(3) | 2577 | 93− | 67− | 5+++ | 2+++ | 3+++ |
| MEXF | | | | | | | |
| 276L | *(3) | 1270 | 95− | 88− | 30+ | 4+++ | 5+++ |
| 394NL | *(3) | 1732 | 99− | 59− | 3+++ | 2+++ | 2+++ |
| 462NL | *(3) | 2593 | 102− | 107− | 20++ | 2+++ | 2+++ |
| 514L | *(3) | 2643 | 108− | 90− | 3+++ | 2+++ | 3+++ |
| 520L | *(2) | 1274 | 102− | 83− | 8++ | 2+++ | 4+++ |
| OVXF | | | | | | | |
| 1619L | *(6) | 2719 | 98− | 88− | 2+++ | 1+++ | 1+++ |
| 899L | *(3) | 1728 | 100− | 94− | 10++ | 3+++ | 4+++ |
| OVCAR3 | *(3) | 2655 | 109− | 103− | 78− | 4+++ | 5+++ |
| PAXF | | | | | | | |
| 1657L | *(3) | 1387 | 96− | 92− | 40+ | 6+++ | 5+++ |
| PANC1 | *(2) | 2255 | 105− | 98− | 33+ | 3+++ | 4+++ |
| PRXF | | | | | | | |
| 22RV1 | *(3) | 1615 | 85− | 80− | 24++ | 2+++ | 2+++ |
| DU145 | *(3) | 2723 | 102− | 94− | 4+++ | 1+++ | 1+++ |
| LNCAP | *(3) | 2987 | 78− | 56− | 4+++ | 3+++ | 3+++ |
| PC3M | *(3) | 3976 | 95− | 91− | 4+++ | 1+++ | 1+++ |
| PXF | | | | | | | |
| 1752L | *(3) | 2522 | 99− | 81− | 27++ | 5+++ | 3+++ |
| RXF | | | | | | | |
| 1781L | *(3) | 1432 | 96− | 76− | 4+++ | 3+++ | 3+++ |
| 393NL | *(3) | 4185 | 96− | 91− | 5+++ | 1+++ | 1+++ |
| 486L | *(3) | 2308 | 102− | 103− | 81− | 9+++ | 5+++ |
| 944L | *(3) | 2656 | 94− | 103− | 9+++ | 3+++ | 3+++ |
| UXF | | | | | | | |
| 1138L | *(3) | 2814 | 102− | 103− | 3+++ | 2+++ | 3+++ |
| XCL | | | | | | | |
| A431 | *(3) | 2105 | 100− | 94− | 6+++ | 3+++ | 2+++ |

| | | | | | |
|---|---|---|---|---|---|
| active*/total | 0/37 | 0/37 | 26/37 | 37/37 | 37/37 |
| cell lines | 0% | 0% | 70% | 100% | 100% |

XF Xenograft Freiburg derived Cell Line|CL Cell Line;
BXF Bladder,
CEXF Cervix,
CXF Colorectal,
GXF Gastric,
LXF Lung A adeno,
L large cell,
E epidermoid cell,
S small cell;
MAXF Breast,
MEXF Melanoma Xenograft,
OVXF Ovarian Cancer Xenograft,
PRXF Prostate,
PXF Pleuramesothelioma,
RXF Renal,
UXF Uterus Body,
XF Miscellaneous Cancer Xenograft − (T/C = 50) + (30 <= T/C < 50) ++ (10 <= T/C < 30) +++ (T/C < 10),
s single plate result;
ng = No Growth

### Example 25

This example illustrates the effect of several compounds, including an exemplary compound of the present invention, on tumor volume. tumor weight and body weight in mice. Nude CD1 male mice were purchased from Harlan (Israel). Animals were housed under controlled conditions (temperature, humidity) and were given food and water ad libitum. After one week of acclimatization animals were inoculated s.c. with human colon carcinoma cells (HT29) (2,000,000 cell/animals.). Therapy began 8 days after inoculation, at which time the animals were divided randomly into 4 groups (7-8 mice/group) and administered ip 3 times weekly for 4 weeks with the following compounds: Compound (Ia) 30 mg/kg, equimolar sertraline, 5 FU 30mg/kg and saline in a volume of 10 ml/kg body weight. Body weight and tumor volume were measured weekly. Tumor volume is defined as (shortest diameter)²×(longest diameter)×0.5. At the end of the treatment, animals were sacrificed and tumors and organs collected and weighed.

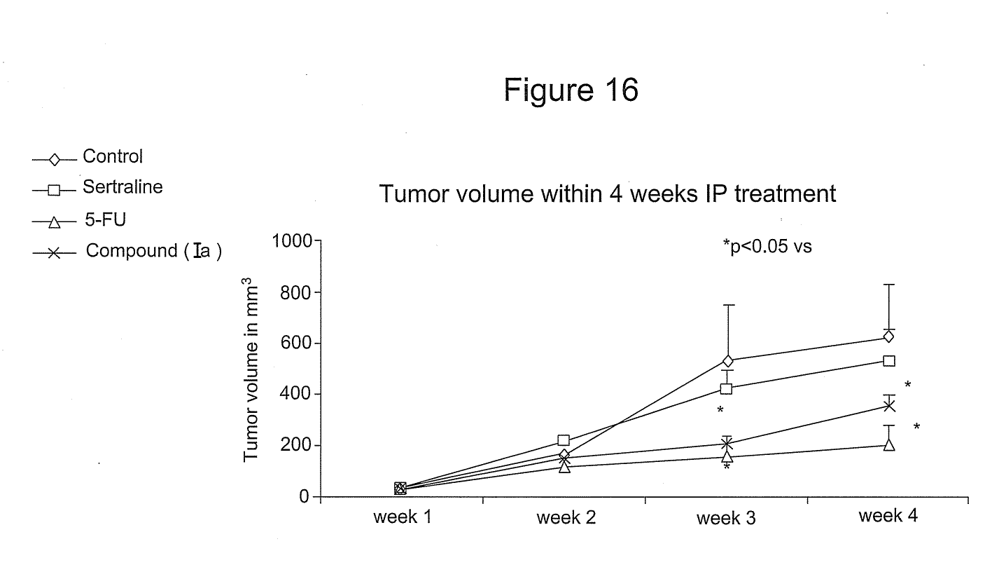

The results with regard to tumor volume are depicted in FIG. 16. The results depicted in FIG. 16 indicate that the tumor volume of mice treated with 5 FU (positive controls) and Compound (Ia) was significantly decreased compared to the control groups after 3 and 4 weeks of therapy.

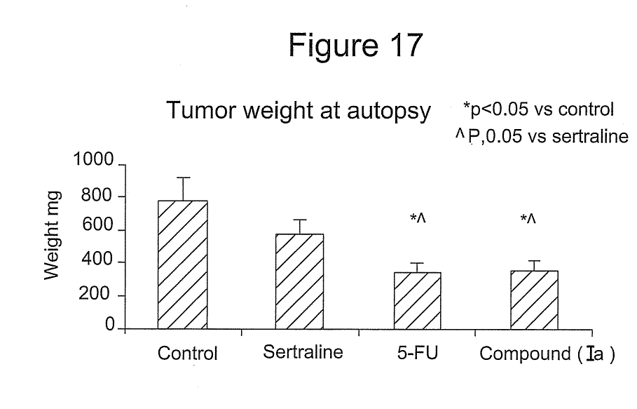

The results with regard to tumor weight are depicted in FIG. 17. The results depicted in FIG. 17 indicate that the tumor weight of mice treated with Compound (Ia) was very similar to the tumor weight of mice treated with 5 FU, suggesting a potential anti-tumor activity for Compound (Ia).

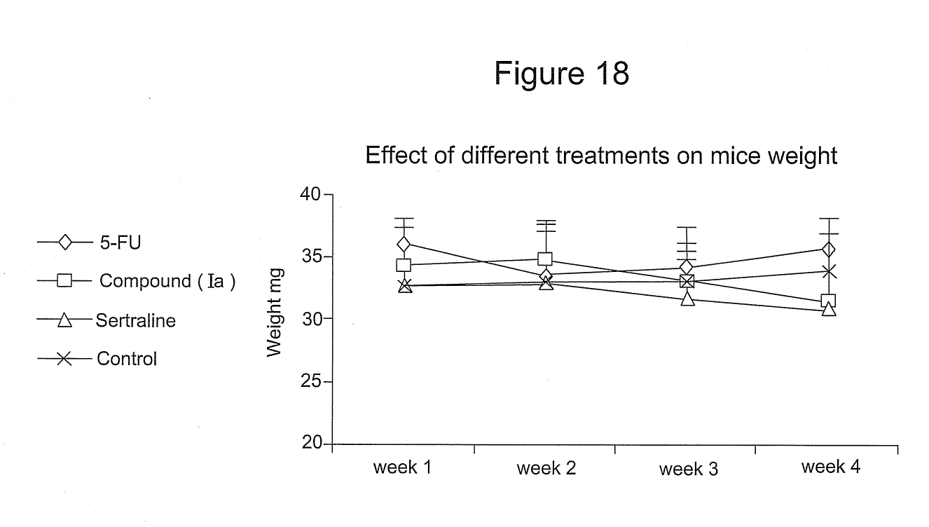

The results with respect to body weight are depicted in FIG. 18. The results depicted in FIG. 18 indicate that Compound (Ia) and sertraline caused a small 10% decrease in body weight compared to 5 FU or controls.

Histopathological assessment of the organs spleen, liver and kidney show a change in capsule fibrosis at random in the sertraline and the Compound (Ia) groups, which may be related to local irritation induced by these agents.

Compound (Ia) showed high efficacy superior to sertraline (resembling 5FU) in inhibiting human colon cancer tumor growth in mice model. Its effect was well tolerated with slight local irritation due to the mode of administration.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of the formula A-L-B (I), wherein:
A is represented by the formula : 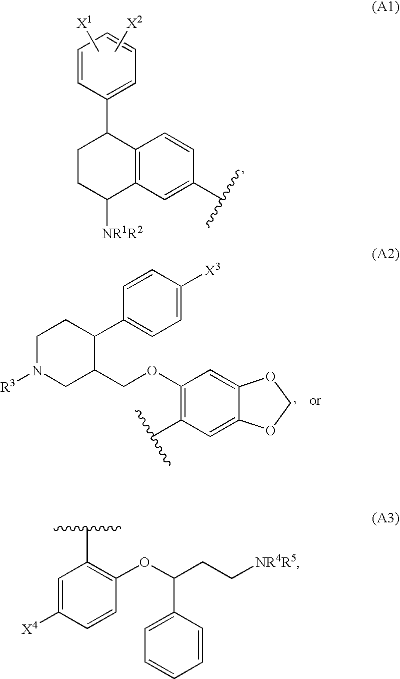
wherein:
R¹, R², R³, R⁴ and R⁵ are the same or different and each is independently a hydrogen or alkyl;
X¹ and X² are the same or different and each is independently a hydrogen, a halogen, haloalkyl, alkoxy, or a cyano;
X³ is a hydrogen, alkyl, alkoxy, haloalkyl, a hydroxyl, a halogen, alkyl thio, or an arylalkoxy;
X⁴ is a halogen, haloalkyl, alkyl, alkoxy, or alkenyl;
L is a linking group comprising two carbon atoms; and
B is an alkyl, alkenyl, alkynyl or aralkyl comprising at least one substituent of the formula Q, wherein:
the alkyl, alkenyl, alkynyl or aralkyl is optionally substituted with one or more halogens, hydroxyl, cyano, nitro, amino, or thiol; and
Q is OR⁶, OC(O)R⁶, C(O)R⁶, C(S)R⁶, CO₂R⁶, C(O) SR⁶, C(O) NR⁶R⁷, C(S)NR⁶R⁷, NR⁶R⁷, NR⁶C(O)R⁷, NR⁶C(S)R⁷, NR⁶C(O)NR⁷R⁸, NR⁶C(S)NR⁷R⁸, NR⁶SO₂R⁷, NR⁶SO₂NR⁷R⁸, SR⁶, SC(O)R⁶, SC(O)NR⁶R⁷, S(O)R⁶, SO₂R⁶, SO₂NR⁶R⁷, or NR⁶SO₂NR⁷R⁸, wherein R⁶, R⁷, and R⁸ are the same or different and each is independently a hydrogen, a C₁₋₆ alkyl, an aryl, an aralkyl, or a pharmaceutically acceptable solubility modifying group; or a salt, ester, or prodrug thereof.

2. The compound of claim 1, wherein B is —(CH₂)ₙ(CHR⁹)ₘQ, —Ar(CH₂)ₙ(CHR⁹)ₘQ, —(CH₂)ₙAr(CHR⁹)ₘQ or —(CH₂)ₙ(CHR⁹)ₘArQ, wherein m and n are the same or different and each is independently from 0 to about 6 provided that m and n are not both zero when B is —(CH₂)ₙ(CHR⁹)ₘQ; Ar is a bivalent aryl; R⁹ is a hydrogen, a C₁₋₆ alkyl, or an aryl; and the Ar, (CH₂)ₙ and (CHR⁹)ₘ are optionally substituted with one or more halogens, hydroxyl, cyano, nitro, amino, or thiol.

3. The compound of claim 1, wherein B is —(CH₂)ₙQ, —(CH₂)ₙArQ or Ar(CH₂)ₙQ, wherein n is from 0 to about 6 provided that n is not zero when B is —(CH₂)/ₙQ.

4. The compound of 1, wherein B is —(CH₂)ₙQ and n=3.

5. (canceled)

6. The compound of claim 1, wherein Q is OR⁶, OC(O)R⁶, NR⁶R⁷, SR⁶ or SC(O)R⁶.

7. The compound of claim 1, wherein A is represented by formula (A1) and wherein X¹ and X² are the same or different and each is a halogen and wherein R¹ and R² are the same or different and each is independently a hydrogen or a methyl.

8. (canceled)

9. (canceled)

10. The compound of claim 7, wherein (A1) is represented by the formula: 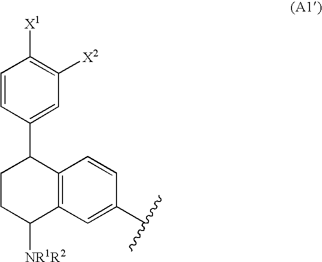
wherein X¹ and X² are the same or different and each is a halogen, and R¹ and R² are the same or different and each is independently a hydrogen or a methyl.

11. The compound of claim 10, wherein X¹ and X² are chlorine, and one of R¹ and R² is hydrogen and the other is methyl.

12. The compound of claim 1, wherein A is represented by formula (A2), wherein R³ is a hydrogen and wherein X³ is a halogen.

13. (canceled)

14. (canceled)

15. (canceled)

16. The compound of claim 12, wherein X³ is fluorine.

17. The compound of claim 1, wherein L comprises a carbon-carbon single bond, a carbon-carbon double bond, or a carbon-carbon triple bond.

18. The compound of claim 1, wherein L is a carbon-carbon triple bond.

19. The compound of claim 1, wherein R⁶, R⁷, and R⁸ are hydrogen.

20. The compound of claim 1, wherein n is from 2 to 4.

21. The compound of claim 1, of the formula: 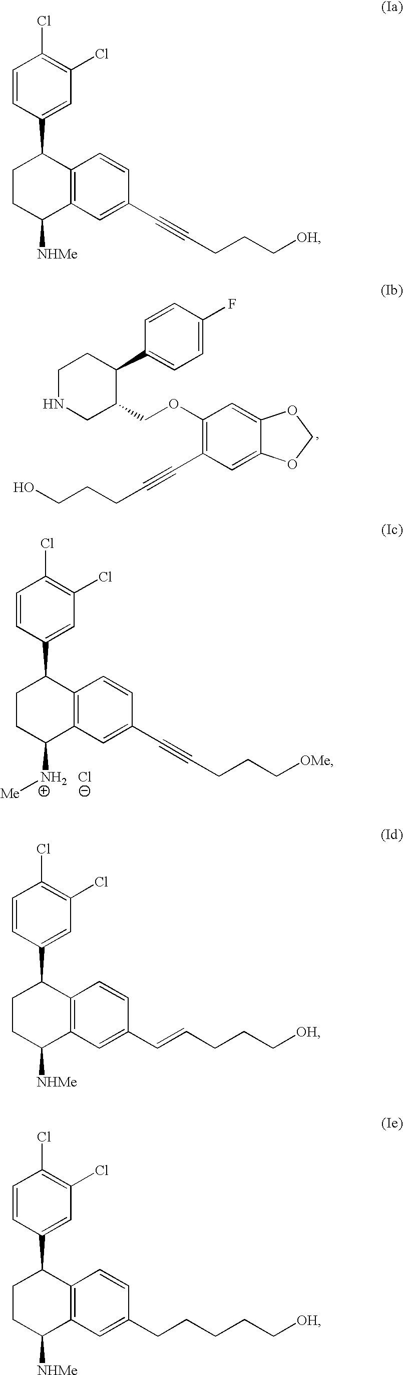 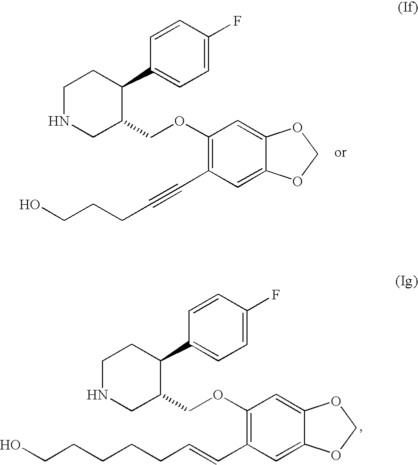
or a pharmaceutically acceptable salt, ester or prodrug thereof.

22. The compound of claim 1, of the formula: 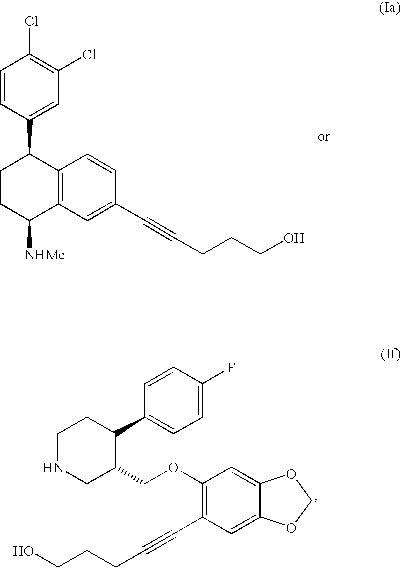
or a pharmaceutically acceptable salt, ester or prodrug thereof.

23. A pharmaceutical composition comprising a pharmaceutically acceptable carrier and a therapeutically effective amount of a compound of claim 1.

24. The composition of claim 23, wherein the composition is orally, topically or parenterally administrable.

25. (canceled)

26. (canceled)

27. A method of treating cancer in a patient, the method comprising administering to the patient a therapeutically effective amount of a compound of claim 1.

28. The method of claim 27, wherein the cancer comprises a multidrug resistant cancer.

29. The method of claim 28, wherein the compound is administered parenterally, orally, or topically.

30. A method of treating a disease or disorder associated with abnormal or undesirable cell proliferation in a patient, the method comprising administering to the patient a therapeutically effective amount of a compound of claim 1.

31. The method of claim 30, wherein the disease or disorder is psoriasis, contact dermatitis, rosacea, seborrheic dermatitis, actinic keratosis, eczema, basal cell carcinoma, melanoma, or atopic dermatitis.

32. The method of claim 30, wherein the disease or disorder is skin cancer, prostate cancer, leukemia, lymphoma, brain cancer, colon cancer, lung cancer, or breast cancer.

33. The method of claim 30, wherein the disease or disorder comprises a non-malignant proliferative disease or disorder.

34. The method of claim 33, wherein the disease or disorder comprises psoriasis hyperkeratosis, reheumatoid arthritis, or scleroderma.

35. The method of claim 33, wherein the disease or disorder is associated with the proliferation of cells of the immune system.

36. The method of claim 35, wherein the disease or disorder is multiple sclerosis, Crohn's disease, ulcerative colitis, rheumatoid arthritis, systemic lupus erythematosis, inflammatory bowel disorder, atopic dermatitis, or contact dermatitis.

37. The method of claim 30, further comprising administering a therapeutically effective amount of a chemotherapeutic agent, other than a compound of any claims 1, wherein the chemotherapeutic agent produces an additive or a synergistic antiproliferative effect.

38. The method of claim 30, wherein the disease is a corticosteroid-resistant hematological cancer.

39. The method of claim 30, further comprising administering a therapeutically effective amount of a corticosteroid, wherein the corticosteroid produces an additive or a synergistic antiproliferative or anti-inflammatory effect.

40. A process for preparing a compound of claim 1, the process comprising:
reacting a compound of the formula: 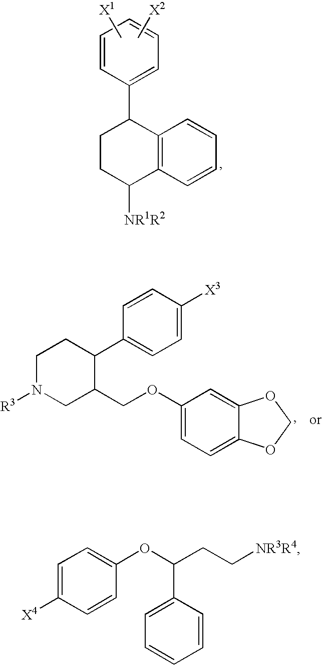
with a halogenating agent to produce a halogenated compound of the formula: 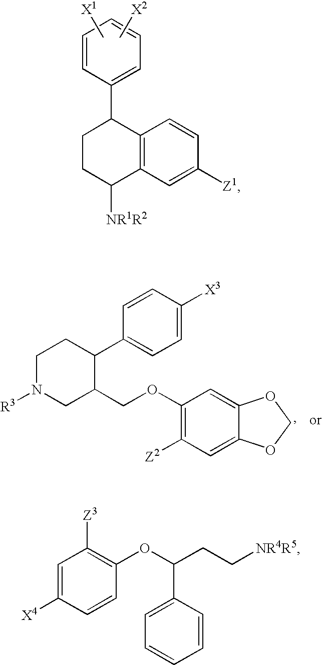
wherein,
Z¹, Z², and Z³ are the same or different and each is a halogen, coupling the halogenated compound with a compound of the formula L-B, wherein L is a linking group comprising a carbon-carbon triple bond and B is an alkyl, alkenyl, alkynyl or aralkyl, to produce a coupling product comprising a carbon-carbon triple bond;
optionally converting the carbon-carbon triple bond in the coupling product into a carbon-carbon double bond or a carbon-carbon single bond,
optionally introducing a pharmaceutically acceptable solubility modifying group, and
optionally converting the coupling product into a pharmaceutically acceptable salt, ester or prodrug thereof.

41. (canceled)

42. The process of claim 40, wherein B is —(CH₂)ₙ(CHR⁹)ₘQ, —Ar(CH₂)ₙ(CHR⁹)ₘQ, —(CH₂)ₙAr(CHR⁹)ₘQ or —(CH₂)ₙ(CHR⁹)ₘArQ.

43. A process for preparing a compound of claim 1, the process comprising:
reacting a compound of the formula: 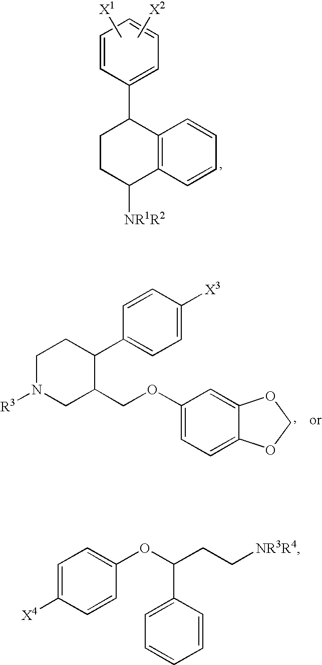
with a formylating reagent to produce a formylated compound of the formula: 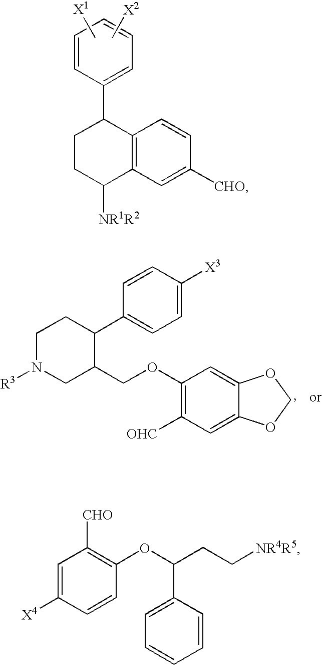
reacting the formylated compound with a reagent capable of reacting with the formyl substituent to produce an alkenyl product of the formula: 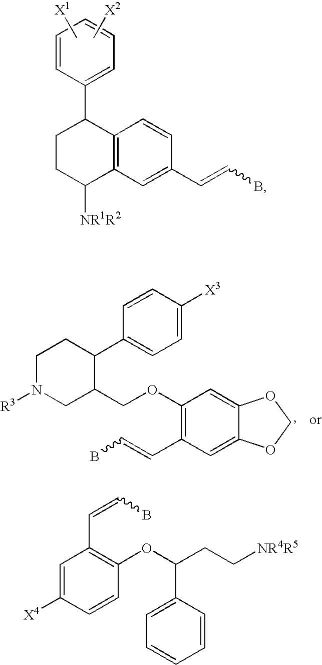
optionally converting the carbon-carbon double bond of the alkenyl product into a carbon-carbon single bond;
optionally introducing a pharmaceutically acceptable solubility modifying group to the alkenyl product; and
optionally converting the alkenyl product into a pharmaceutically acceptable salt, ester, or prodrug.

44. (canceled)

45. (canceled)

46. (canceled)

47. The process of claim 43, wherein B is —(CH₂)ₙ(CHR⁹)ₘQ, —Ar(CH₂)ₙ(CHR⁹)ₘQ, —(CH₂)ₙAr (CHR⁹)ₘQ or —(CH₂)ₙ(CHR⁹)ₘArQ.
